(19) United States
(12) Patent Application Publication
(10) Pub. No.: US 2026/0338691 A1
(43) Pub. Date: Oct. 29, 2026

(54) HIGH GAIN SAMPLING SPECTROMETER

(71) Applicant: ARETE ASSOCIATES, Northridge, CA (US)

(72) Inventors: Paul LUNDQUIST, Longmont, CO (US); Brian DABLE, Canoga Park, CA (US); Karyn APFELDORF, La Canada Flintridge, CA (US)

(21) Appl. No.: 19/115,982

(22) PCT Filed: Sep. 27, 2024
(86) PCT No.: PCT/US24/48799

§ 371 Date: Mar. 27, 2025

Related U.S. Application Data
(60) Provisional application No. 63/540,738, filed on Sep. 27, 2023.

(51) Int. Cl. G01N 21/64

(52) U.S. Cl. CPC G01N 21/6402; G01N 2201/06113; G01N 2201/0636

(57) ABSTRACT

Systems and methods herein provide for identifying an analyte. In one embodiment, a sensor includes a spectrometer and an integrating volume. The integrating volume includes a laser pump resonator configured therein, an interior reflective surface, and an inlet operable to receive a flow of an analyte. The sensor also includes a laser operable to excite the analyte with laser light via the laser pump resonator to generate analyte signature light from the excited analyte. The sensor also includes an optical port configured with the integrating volume, and operable to direct the analyte signature light from the integrating volume to the spectrometer during reflections of the laser light in the laser pump resonator. The spectrometer is operable to identify the analyte based on the analyte signature light.

### CROSS REFERENCE TO RELATED APPLICATIONS

This patent application claims priority to, and thus the benefit of an earlier filing date from, U.S. Provisional Patent Application No. 63/540,738 (filed Sep. 27, 2023), the contents of which are hereby incorporated by reference.

### BACKGROUND

Environmental monitoring of gas, particulates, and aerosols is important for environmental science, health monitoring, and homeland protection. For example, people in general are concerned about environmental substances that can affect human health, whether those substances are from natural phenomena, industrial processes, or malicious attacks. Substances that are embedded in aerosols are particularly difficult to detect through spectral attenuation since the optical properties of a primary aerosol's content may be different than a substance of interest. For example, a chemical threat may be aerosolized within water droplets. And the primary aerosol content of the water may dominate spectral signatures of the substance of interest.

Raman scattering spectral techniques are well known for their ability to detect trace chemical components within a substance because the emission signatures of the trace chemical components have a unique spectral fingerprint that is generated in proportion to the chemical concentration. For example, Raman scattering occurs when light inelastically scatters from molecules. When the scattered photons are lessened in energy by vibrational modes of chemical bonds in a molecule, the emission spectrum provides a spectral signature that can be used for detection of the molecule.

Raman scattering, however, is a weak effect, and detection requires substantial densities of the analyte, long acquisition times, significant pumping radiation, and/or large collection areas. Sensitive detection systems suitable for sustained operation with low power requirements, small volumes, and/or rapid results are challenging to attain because of the low efficiency of Raman scattering.

Amplification techniques, such as resonance Raman or the use of a Surface Enhanced Raman Scattering (SERS) substrate, require that an accumulated sample be potentially collected over a lengthy period of time. While collection over long periods (e.g., 30 minutes to hours) might yield a large sample in complex non-laboratory environments, the sample can be highly cluttered with a potentially complex mix of particles collected over the extended duration. For example, an aerosol plume travelling at 5-10 km/hr may only be present over a point sensor for a few minutes. Collections over extended periods of time not only might add additional clutter but also may delay the delivery of critical, actionable information for those in the vicinity of the sensor when the plume is present.

### SUMMARY

Systems and methods herein provide for identifying an analyte. The embodiments herein enable high-sensitivity spectral detection of scattering from flowing particulates and aerosols. Generally, the embodiments disclosed herein are directed towards detecting components within aerosols where Raman signals can provide information on aerosol content even when “sheathed” within a volume of another material. However, these embodiments may also be used with other types of scattering, including Mie scattering, Brillouin scattering, fluorescence, or the like.

In one embodiment, a sensor includes a spectrometer (e.g., a coded aperture spectrometer) and an integrating volume. The integrating volume includes a laser pump resonator configured therein, and an inlet operable to receive a flow of an analyte. The integrating volume additionally includes a reflective interior surface. The sensor also includes a laser operable to excite the analyte with laser light via the laser pump resonator to generate analyte signature light from the excited analyte. The sensor also includes an optical port configured with the integrating volume, and operable to receive the analyte signature light from interior surfaces of the integrating volume and to direct the analyte signature light to the spectrometer during reflections of the laser light in the laser pump resonator. The spectrometer is operable to identify the analyte based on the analyte signature light. The optical port may include an afocal optical system operable to direct the signature light to the spectrometer. The sensor may be configured with optically reflective fairings operable to block external light from entering the integrating volume from at least one of the inlet, the optical port, or an exhaust port of the integrating volume.

In one embodiment, the sensor increases a signal strength with the laser pump resonator overlapping a particle stream with multiple laser beam passes so as to increase the number of photons collected within an integrating volume with a Raman-inactive, highly reflective coating on the integrating volume's interior reflective surface. A nozzle-in-nozzle device may be used to provide clean, filtered sheath air surrounding a stream of particles that keeps particle flow constrained and away from walls of the integrating volume and the laser pump resonator optics while passing through the laser pump resonator. In some embodiments, a virtual cyclonic concentrator can be added to enrich particle density to yield further signal enhancement.

In some embodiments, the integrating volume comprises an exhaust port operable to exhaust the analyte from the sensor. A blower, vacuum, or other type of device may be used to flow the air/analyte combination through the integrating volume. Such a device may be coupled to the exhaust port to maintain a pressure lower than an external environment. A nozzle configured with the integrating volume may be operable to provide a sheath of clean gas surrounding the flow of the analyte.

In some embodiments, the laser pump resonator comprises two concave mirrors, at least one of which has a laser entrance port. The concave mirrors may have spherical, parabolic or elliptical shapes. In some embodiments, each of the laser pump resonator mirrors includes a spherical reflective surface. In any case, the laser pump resonator mirrors may be configured to substantially confine the laser light within the laser resonator. And the integrating volume may be configured to substantially confine the analyte signature light within the integrating volume (i.e., until the analyte signature light propagates to the spectrometer). In some embodiments the laser pump resonator mirrors may be formed from either metal or glass substrates and coated with a dielectric coating and/or a metal reflective coating. The reflective surfaces of the laser pump resonator mirrors should provide specular reflections with high reflectivity. The laser pump resonator mirrors may be configured with an inlet and an outlet to jointly flow the analyte between the mirrors.

In some embodiments the interior reflecting surface of the integrating volume may be spherical and diffusely reflecting.

In alternative embodiments, the interior reflecting surface of the integrating volume may include both a spherical surface and an elliptical surface conjoined along a circular interface and with both surfaces being designed to provide specular reflection and with the spherical interface additionally including a diffusive patch designed to scatter signature light into the spectrometer. This embodiment may be designed so that center of the spherical portion of the interior reflective surface is aligned to one of the foci of the elliptical portion of the interior reflective surface and the other foci of the elliptical portion of the interior reflective surface is aligned to the diffusive patch. In this embodiment, the laser pump resonator and analyte flow are designed to overlap the laser light and the analyte approximately at a center of the spherical portion of the interior reflective surface.

In some embodiments, the integrating volume includes another optical port operable to propagate the laser light into the laser pump resonator. The other optical port may be configured to propagate the laser light to a center of the flow of the analyte from an acute angle with respect to the inlet.

In some embodiments, the sensor includes a data library comprising data sets of analytes for comparison to spectral data from the spectrometer to identify the analytes. In some embodiments, the sensor includes a library-free deep neural network program to relate spectral data to molecular structures to identify the analytes. In some embodiments, another laser (e.g., having the same or a different wavelength) may be configured to excite the analyte with other laser light in the laser pump resonator.

In one embodiment, a method of identifying an analyte comprises flowing air comprising the analyte into an integrating volume that includes a laser pump resonator configured therein, and exciting the analyte with laser light via the laser pump resonator to generate analyte signature light from the excited analyte. The method also includes directing the analyte signature light from the integrating volume via an interior reflective surface of the integrating volume to a spectrometer during reflections of the laser light in the laser pump resonator and identifying the analyte via the spectrometer based on the analyte signature light.

The various embodiments disclosed herein may be implemented in a variety of ways as a matter of design choice. For example, some embodiments herein are implemented in hardware whereas other embodiments may include processes that are operable to implement and/or operate the hardware. Other exemplary embodiments, including software and firmware, are described below.

### BRIEF DESCRIPTION OF THE DRAWINGS

Some embodiments of the present invention are now described, by way of example only, and with reference to the accompanying drawings. The same reference number represents the same element or the same type of element on all drawings.

FIG. 1 is a block diagram of a sensor for detecting and identifying an analyte in air or other gases, in one exemplary embodiment.

FIG. 2 is a flowchart of a process of detecting and identifying an analyte in air or other gases, in one exemplary embodiment.

FIG. 3 is another block diagram of a sensor for detecting and identifying an analyte in air or other gases, in one exemplary embodiment.

FIG. 4 is a graph comparing Raman spectrums of various coatings that may be used for mirroring an interior surface of an integrating volume, in one exemplary embodiment.

FIG. 5 is a perspective view of a sensor for detecting and identifying an analyte in air or other gases, in one exemplary embodiment.

FIG. 6 is a graphical user interface (GUI) of a tool to design a Raman laser pump resonator, in one exemplary embodiment.

FIG. 7 illustrates a cutaway view of a point sensor system and an interrogation chamber of an integrating volume, in one exemplary embodiment.

FIGS. 8-11 illustrates the spherical and elliptical surfaces of an integrating volume and reflections of scattered analyte signature light, in one exemplary embodiment.

FIG. 12 illustrates a flow system within the sensor system, in one exemplary embodiment.

FIG. 13 illustrates virtual cyclonic concentrator (VCC) concentration bands, in one exemplary embodiment.

FIGS. 14 and 15 illustrate laser pump resonator configurations of an integrating volume, in one exemplary embodiment.

FIG. 16 shows an integrating volume and a coded aperture spectrometer, in one exemplary embodiment.

FIG. 17 is a cross sectional view of an analyte injection inlet, a pump laser injection port, laser pump resonator mirrors, interior reflective surface, and a spectrometer fixture coupled to an optical aperture, in one exemplary embodiment.

FIGS. 18 and 19 show a two dimensional (2D) slice of a Reynolds number of flow at a center of an integrating volume, in one exemplary embodiment.

FIGS. 20 and 21 illustrate a laser pump resonator design, in one exemplary embodiment.

FIGS. 22-24 illustrate laser pump resonator intensity profiles, in some exemplary embodiments.

FIGS. 25 and 26 illustrate mirror design specifications of top and bottom mirrors of a laser pump resonator, in some exemplary embodiments.

FIGS. 27 and 28 illustrate multi-laser inputs to an integrating volume, in some exemplary embodiments.

FIG. 29 illustrates a block diagram of a pump laser injection prism system, in one exemplary embodiment.

FIG. 30 illustrates a block diagram of a laser pump resonator converging a laser to reduce an injection laser aperture on a mirrored surface, in one exemplary embodiment.

FIG. 31 depicts one illustrative cloud computing system operable to perform the above operations by executing programmed instructions tangibly embodied on one or more computer readable storage mediums.

### DETAILED DESCRIPTION OF THE DRAWINGS

The figures and the following descriptions illustrate specific exemplary embodiments. It will thus be appreciated that those skilled in the art will be able to devise various arrangements that, although not explicitly described or shown herein, embody certain principles and are included within the scope of the embodiments. Furthermore, any examples described herein are intended to aid in understanding the embodiments and are to be construed as being without limitation to such specifically recited examples and conditions. As a result, the embodiments are not limited to any of the examples described below.

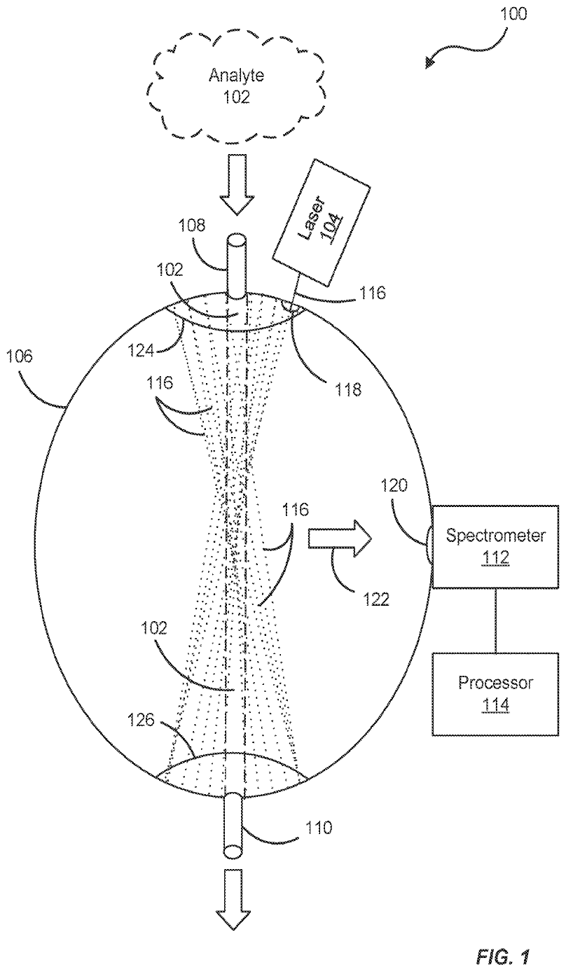

FIG. 1 is a block diagram of a sensor 100 for detecting and identifying an analyte 102 in air or other gases, in one exemplary embodiment. In this embodiment, a sensor 100 comprises an interior reflective surface within an integrating volume 106, a laser 104 (e.g., a diode laser), a spectrometer 112, and a processor 114. The integrating volume 106 comprises a plurality of ports including an inlet 108, an exhaust port 110, an optical port 118, and an optical port 120. The integrating volume 106 also comprises a laser pump resonator configured within the integrating volume 106. For example, the laser pump resonator may be configured from resonator mirrors 124 and 126 at inlet an exhaust portions of the integrating volume 106 (e.g., proximate to the inlet 108 and the exhaust port 110).

The inlet 108 is operable to flow a gas, such as air, comprising an analyte (i.e., gas/analyte 102) into the integrating volume 106. For example, the integrating volume 106 may be configured with a fan, a blower, or some other pump like device (collectively referred to herein as a blower) that is operable to flow a gas comprising the analyte 102 through the integrating volume 106. The blower may flow the gas/analyte 102 into the integrating volume 106 through the inlet 108 via an aperture in the resonator mirror 124 of the laser pump resonator, and out of the integrating volume 106 through the exhaust port 110 via an aperture in the resonator mirror 126 of the laser pump resonator.

In some embodiments, a blower is coupled to the exhaust port to maintain a pressure lower than the external environment. In some embodiments, the flow of the gas/analytc 102 may be sheathed with a clean gas (e.g., air) as the gas/analyte 102 flows through the integrating volume 106. Such may have the effect, among other things, as directing the gas/analyte 102 from the inlet 108 to the exhaust port 110 such that the gas/analyte 102 does not occupy the entirety of the interior of the integrating volume 106 and does not contaminate the interior reflecting surface of integrating volume 106.

The laser 104 is operable to direct laser light 116 into the laser pump resonator. The laser light 116 is directed towards the approximate center of the gas/analyte 102 within the integrating volume 106. For example, the laser 104 may be optically coupled to an optical port 118 configured with the integrating volume 106. The optical port may be configured proximate to the inlet 108 such that the laser light 116 propagates towards a central region of the interior of the integrating volume 106 at an acute angle with respect to the flow of the gas/analyte 102 in the integrating volume 106. The resonator mirrors 124 and 126 of the laser pump resonator are configured to contain the laser light 116 within the laser pump resonator such that the laser light 116 does not “walkoff” the resonator mirrors 124 and 126 into the remaining portions of the integrating volume 106.

The laser pump resonator provides interactions with the gas/analyte 102 near the relative center of the integrating volume 106. These multipass laser light interactions with the gas/analyte 102 excite the molecules of the analyte and provide an integrating effect that results in a stronger analyte signature light. For example, the laser light 116 interacts with and stimulates the molecules of the analyte with multiple passes which, in turn, generates an analyte signature light 122. And, with more interactions, the generated analyte signature light 122 becomes more pronounced.

The reflective surface of the integrating volume 106 may be configured with a reflective material such that the signature light 122 may reflect off the interior reflective surface of the integrating volume 106 multiple times to the spectrometer 112. For example, in some embodiments, the interior reflective surface of the integrating volume 106 may be coated with an electrochemically plated, diffusely reflective, gold-metallic coating.

The spectrometer 112 is optically coupled to the integrating volume 106 via the optical port 120. The spectrometer 112 is operable to collect the generated analyte signature light 122 such that the analyte in the gas/analyte 102 can be identified. For example, the spectrometer 112 may include processing capabilities that illustrate the spectral constituents within the gas/analyte 102 such that the analyte can be identified. This may include comparing spectral datasets of analytes that have been excited with lasers in other measurements, or comparison spectral datasets of analytes that have been obtained from simulations. When the laser 104 excites the gas/analyte 102 where it generates analyte signature light 122, the spectrometer 112 can compare the spectroscopy results to the datasets to identify the particular analyte within the gas/analyte 102. In some embodiments, a processor may be used to implement a deep neural network operable to relate spectral data to molecular structures to identify the analytes.

In some embodiments, the integrating volume 106 may be configured from two concave reflectors that are conjoined to form an enclosure with the reflective portions of the integrating volume 106 residing on the interior of the of the integrating volume 106. In some embodiments, optically reflective fairings are configured with the sensor 100 to block light from entering the integrating volume 106 from at least one of the inlet 108 and the exhaust port 110.

In some embodiments, the integrating volume 106 may be configured as spheres, whereas other embodiments of the integrating volume 106 may include two conjoined reflectors, one of which may be generally spherical with the other being generally elliptical (e.g., having two different foci). Examples of such are shown and described below. As such, the term “integrating volume” is intended to describe an enclosure in which a laser pump resonator is configured therein, and is not intended to be limited to any particular shape as such may be a matter of design choice. Additionally, the terms “mirror” and “reflector” may be used herein interchangeably, though a reflector may support diffuse reflections or specular reflections. Mirrors have specular reflections. And the invention is not intended to be limited to any number of lasers being used, as some of the embodiments disclosed below described multiple lasers propagating laser light at the same or different wavelengths. Other exemplary embodiments are shown and described below.

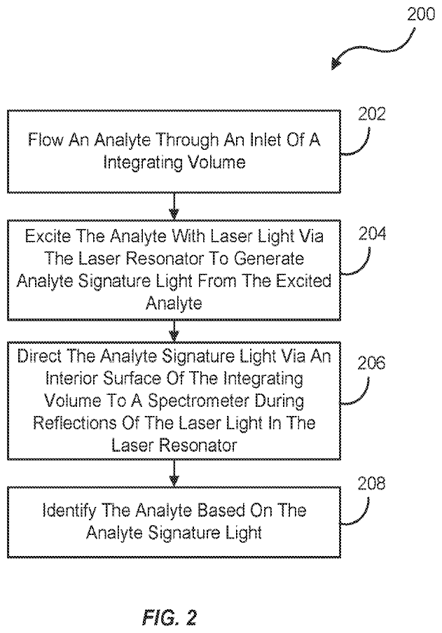

FIG. 2 is a flowchart of a process 200 of detecting and identifying an analyte in air or other gases, in one exemplary embodiment. In this embodiment, the process initiates when a fan of the sensor 100 begins flowing an analyte through an inlet 108 of an integrating volume 106 of the sensor 100, in the process element 202. In some embodiments, the fan is located near an exhaust port 110 of the sensor 100 so as to avoid any residue build-up of the analyte within the integrating volume 106. Additionally, the flow of the analyte through the integrating volume 106 may be sheathed in clean air. For example, another air intake may include a filter to direct filtered air to the integrating volume 106 to sheath the gas/analyte 102 as it flows through the integrating volume 106 to the exhaust port 110.

As the gas/analyte 102 flows through the integrating volume 106, the laser 104 propagates laser light into a laser pump resonator in the integrating volume 106 (e.g., the resonator mirrors 124 and 126) to excite the analyte within the integrating volume 106 and produce the analyte signature light 122, in the process element 204. For example, the laser 104 may be configured proximate to the inlet 108 to propagate laser light into the integrating volume 106 such that it reflects multiple times within the laser pump resonator, generating the analyte signature light 122 in the process. The integrating volume 106 then directs the analyte signature light 122 from the integrating volume 106 to a spectrometer 112 via at least one reflection from an interior reflective surface of the integrating volume 106 during the reflections of the laser light in the laser pump resonator, in the process element 206. And the spectrometer 112 analyzes the analyte signature light 122, in the process element 208, to identify the analyte.

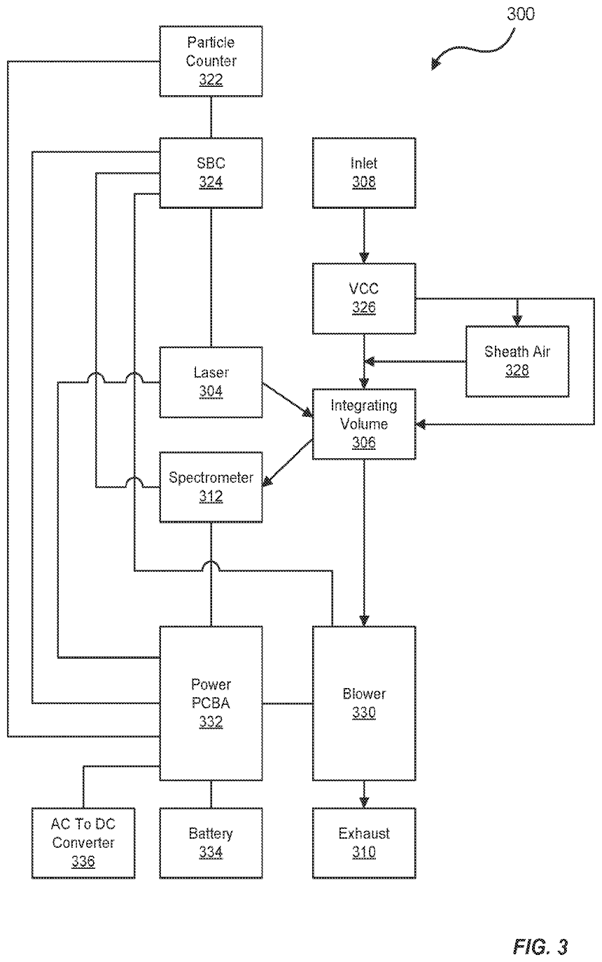

FIG. 3 is another block diagram of a sensor system 300 for detecting and identifying an analyte in air or other gases (i.e., an analyte/gas combination), in one exemplary embodiment. In this embodiment, the sensor system 300 combines aerosol sampling with a virtual cyclonic concentrator (VCC) 326 to enrich particles over a relatively wide size range and to deliver them for sampling within an integrating volume 306, the interior of which may be coated with Infragold® or other suitable materials. The sensor system 300 is considered a “point” sensor system in that it is relatively compact and portable such that it can be placed at almost any location to identify analytes in the air.

The analyte is pulled into the sensor system 300 through an inlet 308. The analyte may be sheathed with clean air. More particularly, a blower 330 flows the analyte/gas combination through the inlet 308 and into the VCC 326, and sheath air module 328 sheaths that flow with clean air, surrounding the analyte/gas combination as it passes through the integrating volume 306. The blower unit 330 ultimately exhausts the analyte/gas combination through an exhaust port 310.

Within the integrating volume 306, Raman emission may be produced by an excitation laser 304. For example, the laser light of the laser 304 may be propagated at an acute angle with respect to the inlet 308 to a laser pump resonator within the integrating volume 306 where it may generate up to 25 times more analyte signature light 122 than would be generated by a single pass of laser light 116. Because excitation occurs throughout the particle laser exposure time within the integrating volume 306, each particle may be sampled several times as it passes through the laser light 116. A separate particle counter 322 may be included to correlate particle counts with signals from the analyte signature light 122.

The sensor system 300 utilizes the integrating volume 306's interior reflective surface to collect analyte signals from a large span of emission angles into a spectrometer 312 coupled to an exterior port of the integrating volume 306. In some embodiments, Raman generated light may be optically coupled to the spectrometer 312 (e.g., via a fiber optic cable), which may be selected to have a large core for increased optical throughput (i.e., etendue). In other embodiments a high-throughput (i.e., high-etendue) free-space optical coupling may be used with a high throughput spectrometer 312. For example, in some embodiments, coded aperture spectrometers may be used to provide much greater optical throughputs than many conventional spectrometers, resulting in an increased spectral signal to noise ratio.

Also in FIG. 3, the sensor 300 includes a power distribution system (i.e., power printed circuit board assembly 332, or ‘PCBA”) that provides AC and/or DC power for the components of the sensor 300 (e.g., from an AC power source, a battery 334, or an AC to DC converter 336). A single board computer (SBC) 324 (e.g., running a Linux Operating system) may be used to provide command and control of the sensor 300. The SBC 324 may include an analyte detection algorithm for real-time autonomous operation. The sensor 300 generally requires no consumables, except for the optional collection of the particles for secondary analysis, if desired. The sensor 300 may also perform continuous monitoring with frequent and regular responses to detections. For example, the sensor 300 may include a communications module with the SBC 324 to detect and respond to short duration plumes (e.g., with minute time sensitivity or less) that may occur in outdoor environments.

The embodiments herein provide an ability to continuously monitor ambient air through the inlet 308, with a reporting-time that is long enough to maximize the amount of Raman scattering captured over read noise and dark current of the spectrometer 312. But the embodiments may also make several measurements while a plume is detectable by the point sensor 300.

The integrating volume 306 allows Raman scattering emissions from almost all angles to be collected. For example, the integrating volume 306 may be configured with a highly reflective interior surface. The laser 304 may excite the gas within the integrating volume 306 such that Raman scattering occurs in generally all directions within the integrating volume 306. The integrating volume 306 may thus provide a more complete collection of the Raman scattered light as emissions reflect inside the integrating volume 306 until the emissions reach an optic or aperture for collecting light with the spectrometer 312. In one embodiment, the interior surface of the integrating volume is coated in Infragold®, or some other electrochemically plated, diffusely reflecting, gold-metallic coating. Infragold® has a 95% reflection. Thus, Infragold® is not the most reflective material that could be used to coat the integrating volume 306, but Infragold® is Raman inactive. More reflective coatings, such as Spectralon or Spectraflect, are Raman active and may add interference into each measurement.

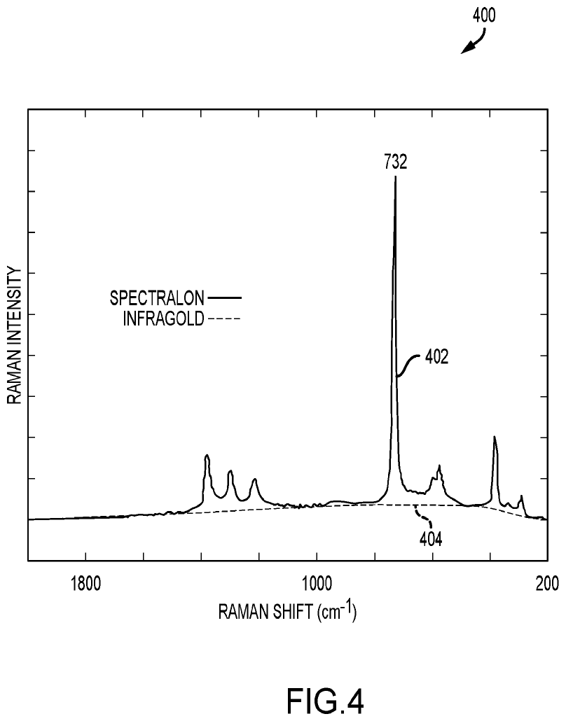

FIG. 4 is a graph 400 comparing Raman spectrums of various coatings that may be used for mirroring an interior surface of an integrating volume, in one exemplary embodiment. Here, the graph 400 compares the Raman intensity versus the Raman shift for Infragold® 404 and Spectralon 402.

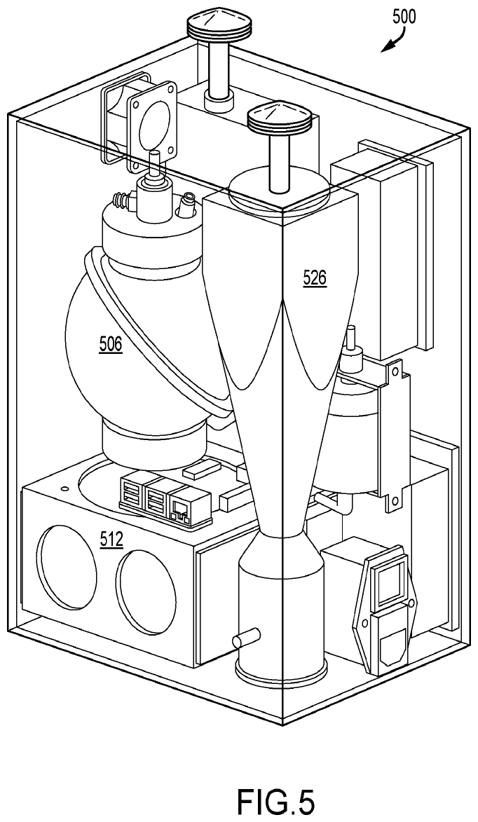

FIG. 5 is a perspective view of one embodiment of a sensor 500 for detecting and identifying an analyte in air or other gases, in one exemplary embodiment. A virtual cyclonic concentrator 526 enriches input particle concentrations by about 5 times over a broad range of particles, e.g., those that are nominally between 0.01-10 μm. A “nozzle-in-nozzle” design to control the air stream and avoid contamination. For example, a blower system 512 may be configured to pull an analyte flow through the integrating volume 506. The blower system 512 may also be used to pull air through a filter so as to provide a sheath air around the analyte flow. The blower system 512 may be configured at the exhaust port of the sensor 500 so as to prevent residue of analytes in the integrating volume 506.

Particles enter the integrating volume 506, one that is nominally 4″ in diameter and is configured with a many-pass laser pump resonator, in this embodiment. The integrating volume 506 directs laser light along a particle stream and focuses a frequency-stabilized 785 nm diode laser light onto the particle stream, maximizing Raman scattering events and increasing signal strength by about 25 times (e.g., relative to single pass excitation). The Raman emission scatters within the Raman-inactive-coated integrating volume 506 prior to exiting to a spectrometer (not visible in FIG. 5), providing a more complete capture of photons than can be achieved with conventional transmission or reflection spectroscopy, and yielding about a 10× increased signal (e.g., relative to detection without the interior reflective surface on the integrating volume). Together, these enhancements boost a Raman signals strength by about 1250 times (e.g., relative to laser excitation of the analyte flow and detection from a spectrometer).

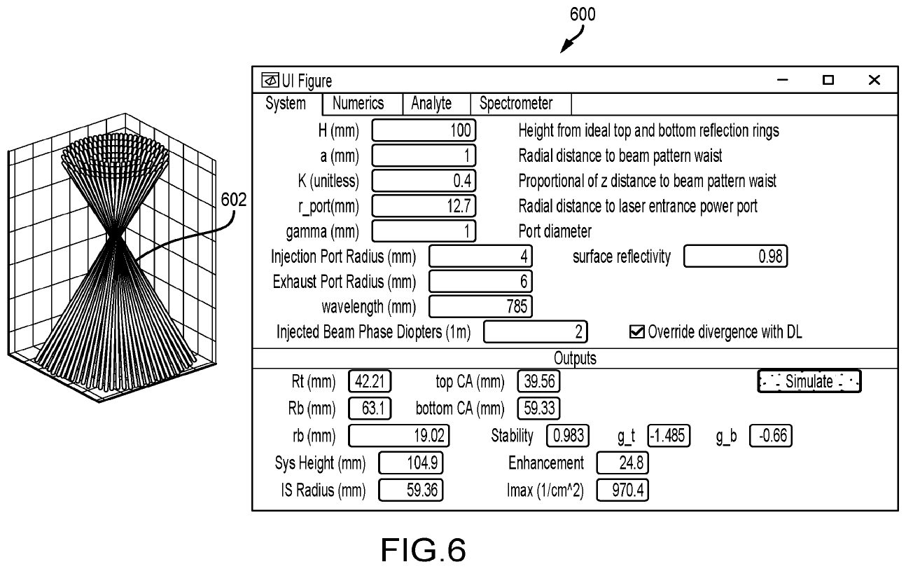

FIG. 6 illustrates a laser pump resonator design tool 600 with pump resonator parameters incorporating a nominal 450 mW laser. With this design, the point sensor system 500 is able to detect a 0.05-micron minimum particle size of weakly scattering chemical warfare agents. The integrating volume 506 enhances collection of spectral signals since Raman scattering that is scattered from the integrating volume 506's interior can still be received by the spectrometer (e.g., hidden behind the VCC in FIG. 5) The airflow is controlled by an in-line blower (512), which pulls a relatively high volume of air through the sensor device 500 and exhausts that volume after interrogation. In some embodiments an increase in particle concentration (e.g., about 5 times) may also be obtained from a virtual cyclonic concentrator 526 in the airstream.

In some embodiments, particulates are collected onto a coarse electrostatic filter material for optional secondary analysis, following positive detection. A particle counter (e.g., laser particle counter such as particle counter 322 of FIG. 3) may be included as an auxiliary channel to assist with false alarm mitigation and to modulate collection time. The integrating volume 506 may boost signal strength but it may also tame signature variability. While the Raman signal for a single particle has a nonmonotonic dependence on particle size due to the complex Morphology-Dependent Resonance (MDR) features, integration over many particles may provide a smoothing function. For example, the integration over many particles may provide a net ensembled Raman signal that is roughly proportional to the material's total mass, with each particle contributing in proportion to its volume. Longer integration times (e.g., several minutes) can be used to further boost signal strength.

To maximize Raman excitation within the integrating volume 506, the design tool 600 may be used to quantify multi-pass laser interaction amplification as a function of design parameters. The design tool 600 is used to design the Raman pump resonator 602 and numerically compute a radial energy distribution of a two-mirror laser pump resonator through optical raytracing. And the pump injection location is specified as an off-axis entrance port. The design tool 600 may be used to calculate the circulating intensity spatial distribution and resonator stability parameters. The design tool 600 may also allow design optimization of pump power overlap with an analyte particulate stream. Computations indicate that the net effect on laser power may be an approximate 25-multiplicative increase over a single laser line pass through the interrogation region for a nominal 4″ diameter sphere design.

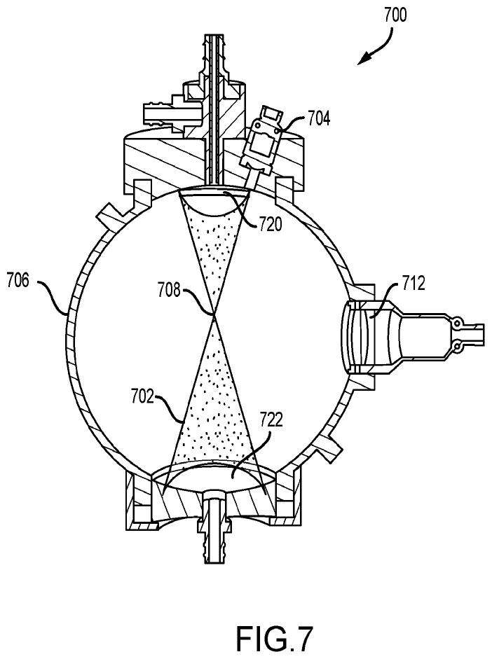

FIG. 7 illustrates a cutaway view of a point sensor system 700 and the interrogation chamber 710 of an integrating volume 706. The integrating volume 706 provides a multi-pass pump laser pump resonator 702 configured with the resonator mirrors 720 and 722. The laser 704 fires laser light towards the resonator mirror 722, which in turn reflects the laser light to the resonator mirror 720. The laser light is largely contained within the laser pump resonator 702 so as to not walk off the resonator mirrors 720 and 722. The resonator mirrors 720 and 722 may be configured in such a way so as to converge/concentrate the laser light to a region 708 near a center of the integrating volume 706.

In one embodiment, a frequency-stabilized 785 nm diode laser 704 having a relatively narrow bandwidth holds the emission wavelength constant over a wide range of temperatures which aids in matching spectral features over different environments and collections. Some factors for laser selection may include: (1) power flux of the light that the laser can produce; (2) probability for fluorescence emission interference based on the source wavelength; (3) dependence on the source wavelength to fully excite scattering in relevant particle sizes; (4) power requirements; (5) laser spectral linewidth and stability with changing environmental conditions; (6) laser source divergence or beam quality; and (7) laser polarization.

While a UV and visible light sources have advantages in stronger Raman emission intensity, fluorescence could be an issue with a visible laser. A near infrared (NIR) laser like the 785 nm laser allows full illumination of encapsulated particles. While a NIR laser should excite through the center 708 and yield a stronger emission of a target chemical that has been encapsulated, it should also perform better in terms of fluorescence interference.

In some embodiments, sensing is performed by integrating spectral signatures over short times that can be averaged across many measurements while an aerosol cloud is in the vicinity of the sensor 700. The high resolution of Raman allows integration of signals over seconds to build a relatively recent snapshot of air that can be separable into components, as described in greater detail below. This resolution is also able to take advantage of trending and background adaptation to identify target compounds and adapt to local changes in the aerosol environment that come due to environmental changes.

In one embodiment, the pump resonator 702 of the interrogation chamber 710 in the sensor system 700 increases the circulating intensity Iₗₐₛₑᵣ by a factor of 25×. As an example, the Raman cross-section for a Soman nerve agent GD has been computed for a 731 cm⁻¹ Raman peak using a 213 nm laser to be 7.68×10⁻²⁸ cm²/sr/molecule. Scaling for the λ⁻⁴ dependence on cross-section, the cross-section can be estimated as reduced to 4.16×10⁻³⁰ cm²/sr/molecule in an embodiment using a 785 nm laser source. Based on the volume, molecule mass, particle density, and assuming a 42 ms transit time through the pump resonator 702, it can be estimated that a 450 mW laser source would result in Raman emission of 250 photons from a 1 μm particle. Much of the emission would be collected through the use of the interrogation chamber 710 with the notional designed many-pass pump resonator 702.

The number of particles per liter that may be present for a given size can be described as

$${n}_{particle} = \frac{{concentration}_{particle}}{\frac{4}{3} π {r}_{particle}^{3} \times {ρ}_{particte}} ,$$

where concentration_{particle} is the mass density of the particulate distribution, ρ_{particle} is the density of a single particle, r is the radius of a single particle.

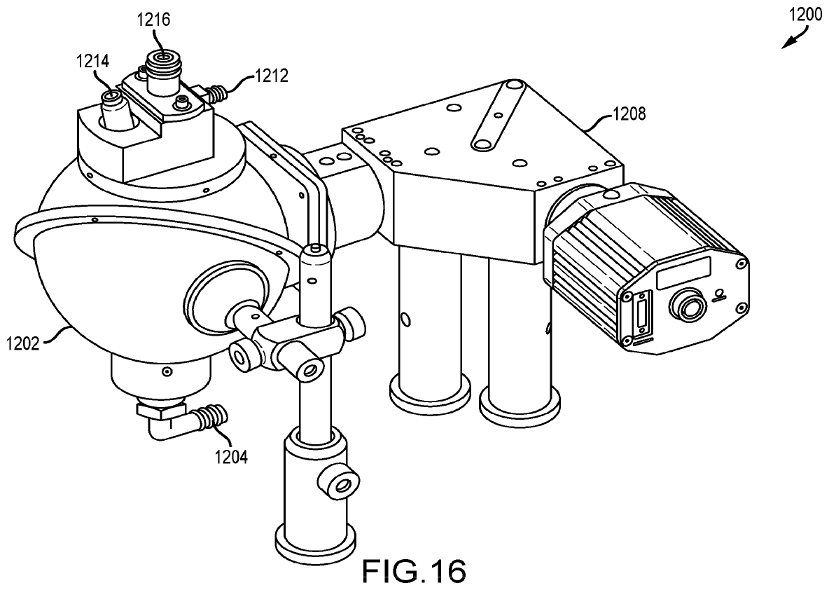

n_{particle} can be multiplied by I_{particle}, where I_{particle} is the number of photons scattered from each particle to record a total number of photons scattered. The enhancement from the VCC (e.g., VCC 326 of FIG. 3) can be accounted for because the VCC yields a larger number of particles in the interrogation region 710 at a lower flow rate than was initially sampled. This allows estimation of the total number of scattered photons available to detect using each enhancement factor. For example, when integrating for 60 seconds, the number of photons produced from the Soman nerve agent GD particles would be over 9.3 million photons for a given Raman shift from that cross-section. The photons may scatter in all directions inside the interrogation region 710. Because the interior surface of the interrogation chamber 710 is highly reflective in this embodiment, a greater chance exists for these photons to reach the aperture 712 of the spectrometer (e.g., 1208 in FIG. 16). For smaller sized particles, at the same concentration, the total mass of chemical measured would not change because there would be more, but smaller particles. Regardless, smaller particles of equivalent materials generally would emit fewer photons.

When compared to a traditional reflective or transmission Raman instrument design, the embodiments herein yield significantly higher signal returns due to the many-pass pump resonator 702. The pump resonator 702 provides light to a portion of the particulate flow path allowing for a longer time to excite the Raman scattering in each particle. Reflection of Raman scattered light from the interior reflective surface of the integrating volume 706 permits the spectrometer to collect light initially emitted from particulates into a large solid angle.

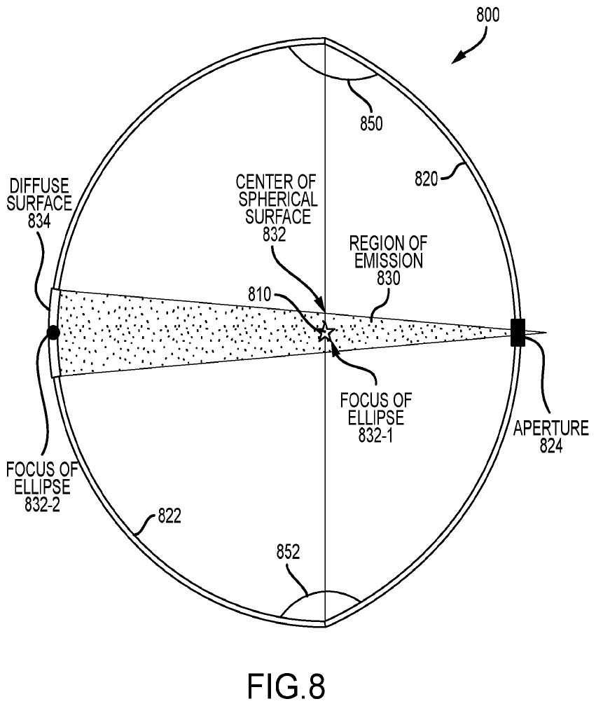

FIG. 8 illustrates an integrating volume 800 with interior reflective surfaces that are spherical and elliptical mirrors 822 and 820 forming an interrogation region 810, comprising specularly reflecting surfaces, in one exemplary embodiment. The integrating volume 800 collects scattered signature light from the analyte and provides one or more surface reflections to direct the scattered light into the entrance of a spectrometer (i.e., aperture 824). The integrating volume 800 is also configured with flow apertures (not shown here) to permit the passage of the analyte. The integrating volume 800 also includes resonator mirrors 850 and 852 that are configured in the integrating volume 800 to provide a laser pump resonator that overlaps between the pumping radiation and analyte flow therewithin.

In some embodiments, the interior reflective surfaces 820 and 822 of the integrating volume 800 provide high reflectivity and have specular reflections. Thus, the interior reflective surfaces 820 and 822 should be comprised of a material having low fluorescence at the selected laser pump wavelengths. Some embodiments may use multiple surface shapes with both specular and diffusely scattering surfaces. Aside from the diffusely scattering patch 834, the interior reflective surfaces 820 and 822 of the integrating volume 800 specularly reflect analyte signature light within the integrating volume 800.

In FIG. 8, the surface 820 is a specularly reflective interior surface that is ellipsoidal in shape, while the other surface 822 is a spherical specularly reflective interior surface with a diffusely scattering patch 834 that is matched to a field of regard of the spectrometer. This approach recirculates analyte signature light to be incident on the diffusely scattering patch 834, which may be used to siphon off the light into the spectrometer (not shown). In some embodiments, a filter may be implemented at the optical coupling to the spectrometer to prevent laser light from entering the spectrometer while permitting analyte signature light to be received from by the spectrometer. The spectral filter may be included within optics of the optical coupling to be effective for light incident on the optical coupling at moth small and large off-axis angles.

This embodiment, while differing somewhat from those above, is generally more efficient than an integrating sphere as, on average, there are fewer interior “bounces” before collection for less total loss before the analyte signature light enters the spectrometer. Additionally, this embodiment reduces the required field of view for the spectrometer permitting more effective spectral filtering of pump laser light to reduce interference with the analysis of the analyte signature light. The interior reflective surface 822 is spherical and the pump resonator (not shown) is configured so that the region of emission is near the center of the radius of curvature for interior reflective surface 822. Most of the surface is specular, but a diffusely scattering patch 834 in the region may be viewable in the field of regard of the spectrometer through the aperture 824. The surface 820, being ellipsoid, may have a foci 832-1 corresponding to the region of emission 830 and the center of the diffusely scattering patch 834. It should be noted that, while one design choice may be to make the spherical surface a hemisphere, other embodiments may include a spherical surface with different proportions of a full sphere where the emission region may still be near the center of the radius of curvature of the spherical interior reflecting surface 820.

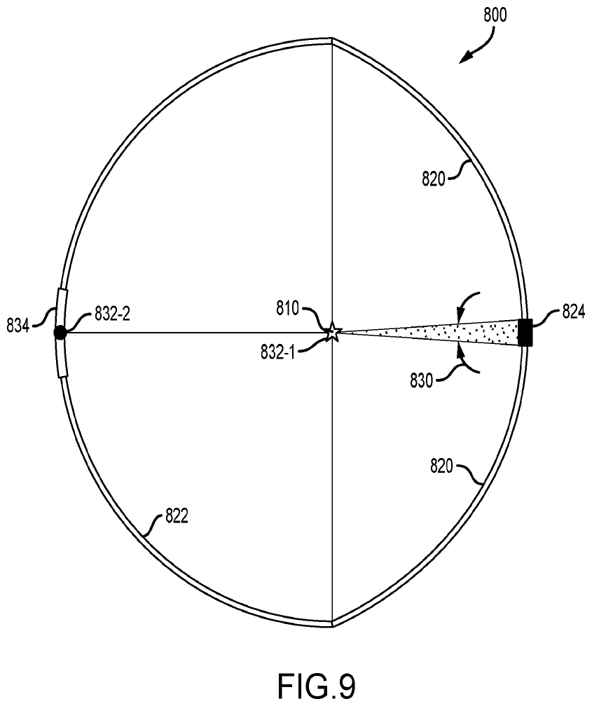

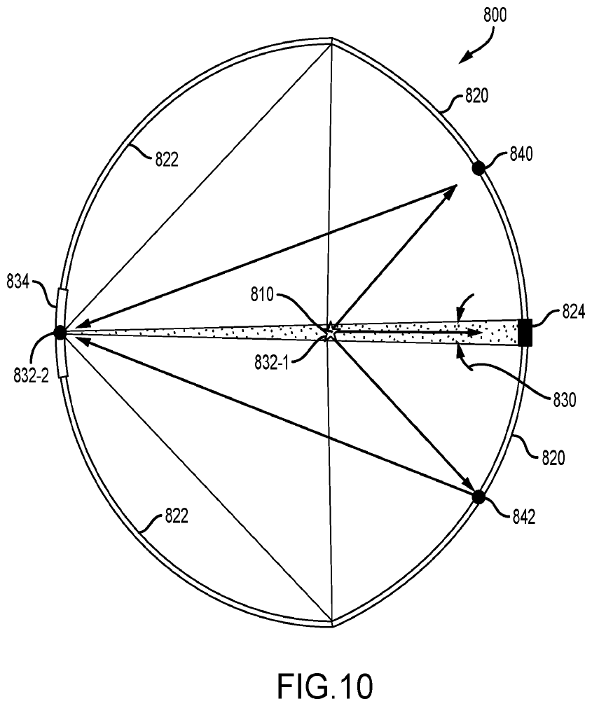

In FIG. 9, FIG. 10, and FIG. 11, the resonator mirrors 850 and 852 are not shown for simplicity. Here, some portion of photons emitted from the interaction region 810 directly reaching the sensor aperture 824 through a solid angle 830 without reflections from the integrating volume 800's interior surfaces. The interaction region is a region where the analyte is being excited by the laser pump resonator and is generally designed to overlap the center of the spherical surface 820.

However, a much larger portion of the photons emitted from the interaction region 810 hit the elliptical surface 820. FIG. 10 shows the path of light from emitted photons from the interaction region to the elliptical interior reflecting surface 820 (e.g., including points 840 and 842). Those photons are then focused to a diffusely scattering patch 834 that overlaps the one of the foci of the elliptical surface 820. Some portion of the photons that hit the diffusely scattering patch 834 scatter into the solid angle 830-1 to the aperture 824 where they are collected by the spectrometer. For example photons traveling a path of point 832-1 to point 840 to point 832-2 to aperture 824 follow this described reflective process to being received by the sensor aperture 824.

FIG. 11 shows the reflective process for light reaching the diffusive patch that scatters back to the elliptic interior reflective surface 840. Photons that are diffusely scattered back to the elliptic surface 820 (e.g., which include points 840 and 842) are imaged by the elliptic surface 820 into the emission region 832-1. But then those photons travel to the spherical surface 822 (e.g., which includes point 844) and is retroreflected back through the emission region 832-1 and is incident back onto the spherical interior reflective surface (e.g., containing point 840). The photons are again reimaged onto the diffusely scattering patch 834, from which some proportion of the light scattered into the sensor aperture 824. As an example of this reflective process, some photons travel a path from the diffusely scattering patch to point 840, through emission region 832-2, to point 844, back through emission region 832, back point 840, and then back to the diffusive patch 832-2. Whenever light is incident on the diffusive patch 832-2, some proportion of that light is coupled into the sensor aperture 824 for analysis by the spectrometer (or any other optical analysis instrument).

The emission from the point 832-1 includes photons that are initially incident on the spherical interior reflective surface 822 (e.g., which includes the point 844). Those photons are reimaged back into the point 832-1 and traverse to the elliptical interior reflective surface 820 where the point 840 is located. The subsequent reflective processes are the same as light that is emitted from 832-1 toward elliptical interior reflective surface 820. While there are paths for non-imaging light fields (e.g., the diffuse light from the diffusely scattering patch 834 that fall onto the spherical interior reflective surface 822 (e.g., where point 844 is located), and the initial photons from the point 832-1 that fall onto the diffusely scattering patch 834), most of the light gets reimaged onto the diffusely scattering patch 834 in a recirculating manner to maximize the light capture from the emission. The diffusive scattering patch 834 acts to outcouple some proportion the light incident upon it to the sensor aperture 824.

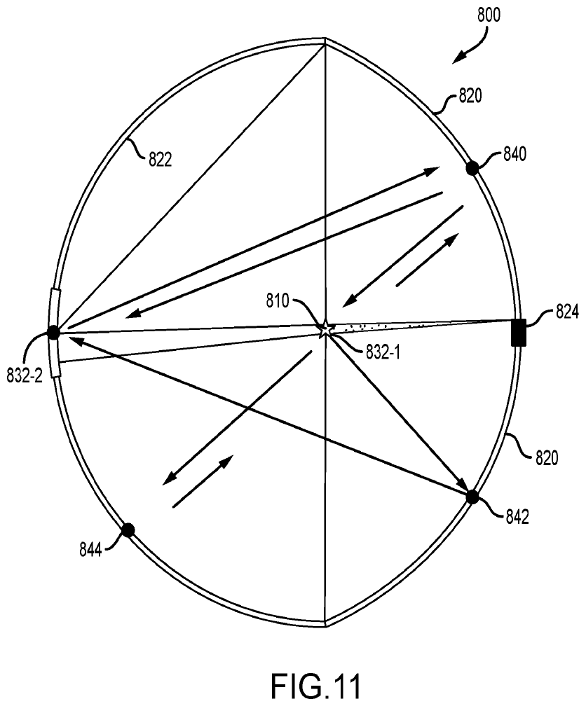

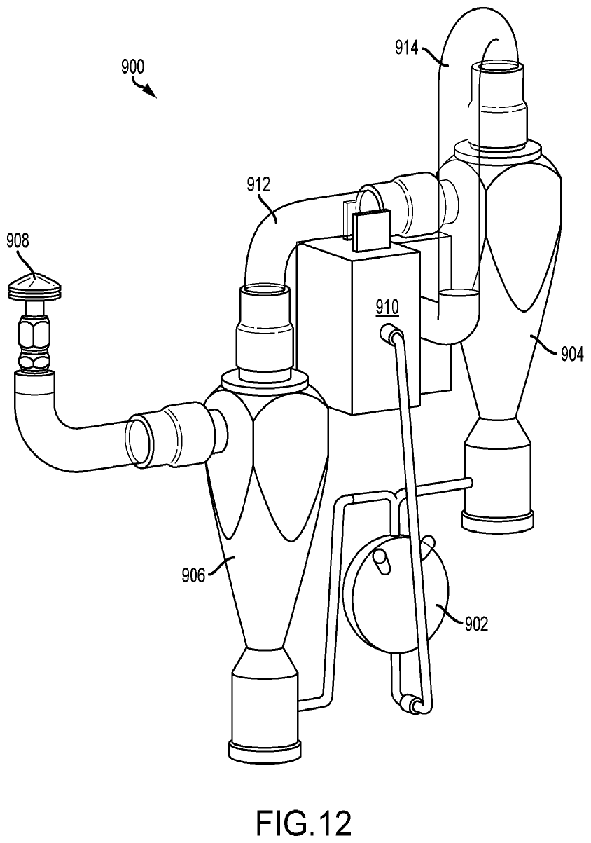

FIG. 12 illustrates a flow system 900 within the sensor system, in one exemplary embodiment. The flow system 900 utilizes a “pull through” flow system whereby the pump 910 (e.g., a fan) draws aerosol particulates through the sensor system. The pump 910 may be placed after the exhaust of the integrating volume. Placing the air draw in this location minimizes the opportunity for prior analyte samples from sticking onto a surface such as a fan blade that may “shake loose” later and function as a noise source or source of false alarm in the sensor system. For example, some particles of an earlier sampling may adhere to surfaces in the pump 910. By placing the pump 910 near the exhaust of the sensor system, these particles would not be observed by the spectrometer as they have already passed through the sensor system. Thus, there would be no inadvertent detections that could trigger an alarm. Additionally, air tubing (e.g., tubes 912 and 914) within the flow system 900 could be made of a material with low electrostatic properties so as to not attract particles that could stick to interior surfaces, loosen over time, and thus produce a false measurement.

Keeping the analyte flow from having opportunities to collect on the interior surfaces in the integrating volume is generally important for minimizing the background noise within the detected signals over long-term use. Should particles collect on surfaces they could introduce a constant background signal and function as a source for specular reflection that may reduce the effectiveness of the sensor system. Therefore, several design considerations can be made such as having aerosol flow within the integrating volume flow vertically in the direction of gravity. Thus, should the flow be broken (e.g., when the sensor system is disabled), gravity could pull particulates to the exhaust port without landing on an interior surface of the integrating volume.

Generally, the particle flow entering the integrating volume from an inlet (not shown here, but similar to inlet 108 of FIG. 1 and inlet 1216 of FIG. 16) will tend to slow down and spread due to the larger volume. To minimize this, the flow should be designed to maintain a low Reynolds number such that the flow will tend to stay laminar. Thus, in one embodiment, a nozzle-in-nozzle design may be implemented such that the particulate flow is surrounded by filtered particle-free “sheath” air via the sheath air module (not shown here, but similar to module 328 of FIG. 3,). For example, the sheath air module may include a high-efficiency particulate absorbing (HEPA) filter to constrain the particle flow in a second stream of air that passes through. The velocity of the flows between the sheath air and the particle flows should match for the flows to seamlessly merge into one larger diameter flow. One benefit of the sheath air is that flow that will spread widest will be the sheath air. This sheath air may not entirely pass through to the exhaust, but it will not contain particles that could cause interference. Additionally, the total flow velocity may be configured in such a way as to not cause turbulence within the integrating volume.

In this embodiment, the flow system 900 comprises a dual virtual cyclonic concentrator (VCC) comprising VCCs 904 and 906. The overall design of these flow components, tubing sizes of the tubes 912 and 914 that connect them together, along with in-line flow restrictors to balance the flow rate at each point, may be designed using computational fluid dynamic (CFD) software. The dual VCC includes an inlet 908 that has an inertial separation cut-point to allow particles up to 10 microns aerodynamic diameter to be aspirated into the system. The particles may then be concentrated in the virtual cyclones to provide a detectable quantity to flow system 900. VCCs can enrich small particles which fall to the bottom cup, where they are withdrawn in the minor flow.

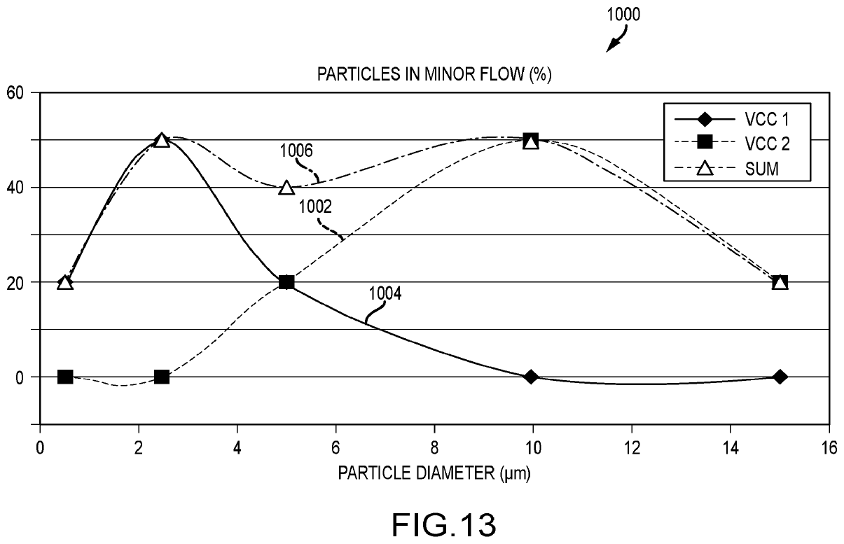

FIG. 13 illustrates VCC concentration bands that are “tuned” by balancing major and minor flowrates, in one exemplary embodiment. Here, a first VCC produces the concentration band 1002 and another VCC produces the concentration band 1004. The sum of these flows is seen in the concentration band 1006. Using two VCCs allows enrichment across the bulk of the target particle size range by delivering the minor flows of each VCC into the interrogation region at between 1% and 10% of each cyclone's inlet flow. The VCC concentration bands 1002 and 1004 may be “tuned” by balancing major and minor flowrates. Lowering the percentage of total flow withdrawn via the minor flow may concentrate smaller particles. Increasing the minor flow withdrawal percentage increases the particle size range that is concentrated. The VCC's particle enrichment performance can then be assessed prior to integration with a Raman spectroscopic subsystem.

Following concentration, the particle streams are combined and directed to the inlet of the integrating volume. A stream of filtered sheath air may be co-introduced around the particle stream as the flow enters the integrating volume. Sheath air acts to constrain the particles (e.g., where the laser from the Raman subsystem can interact) because the tendency of the flows is to slow and begin spreading when there is no longer a constraint from tubing to keep laminar flow. One embodiment of the sensor system directs air exhausted from the integrating region back to the environment from which it was sampled, but away from the inlet to avoid resampling.

In some embodiments, a separate laser particle counter is integrated within the flow, such as particle counter 322 of FIG. 3. The laser particle counter may be configured outside the integrating volume to serve as an orthogonal measurement that can improve the probability of detection (PD) versus the probability of a false alarm (PFA) and modulate response time. Releases or clouds of aerosol add to the ambient aerosol environment, resulting in changes in the number and distribution of particle sizes. An independent laser particle counter can monitor the general particulate level to track and trend particle concentrations at different sizes. In some embodiments, a multiple-channel laser particle counter based on Mie scattering can measure particles between 0.3-10 micron.

In one embodiment, the sensor system has at least 5 separate power domains, likely running from a single common 9-36 V input bus. Total maximum instantaneous power draw may be 80 W, however the average power draw is typically 30-40% of the maximum after an initial warmup and during operation in normal room temperature environments because most of the power draw is from thermoelectric coolers attached to the laser driver and detector. A single battery pack may be used to run the system for 24 hours. But a hot-swap interface may allow for continuous operation during a battery swap. Alternatively or additionally, an AC power supply may be used.

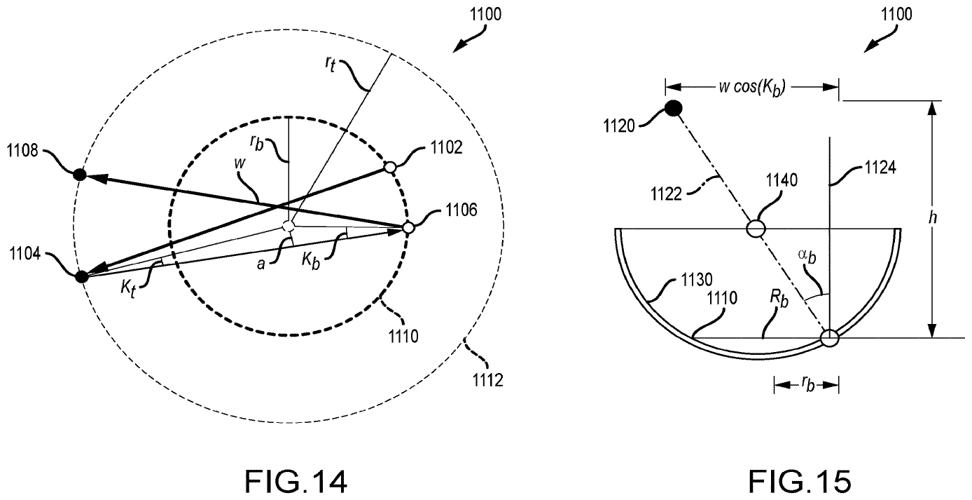

FIGS. 14 and 15 illustrate laser pump resonator configurations of an integrating volume, such as the integrating volume 106 of FIG. 1. More specifically, FIG. 14 shows a view of rays starting from a bottom reflector 1110 from a bottom point 1102 to a top point 1104 of a top reflector 1112, to a bottom point 1106 of the bottom reflector 1110, and to a top point 1108 of the top reflector 1112. And FIG. 15 shows a side view of the bottom reflector 1110 having radius of curvature R_{b} with the center of the radius of curvature being indicated by point 1140. The laser pump resonator is configured to contain the pump laser light within the reflectors 1110 and 1112 such that the laser light does not “walk off” the laser pump resonator and into the integrating volume.

In this embodiment of the pump resonator, the two opposing mirrors 1110 and 1112 have a common axis of rotational symmetry (e.g., a rotational axis). Without loss of generality, the top reflector 1112 is the mirror from which the pump laser light is injected into the resonator through a pump laser injection port (e.g., the aperture 118 of FIG. 1). The radial distance from the rotational axis to the pump laser injection port is given by rₜ. The laser is directed to be incident on the bottom reflector 1110 at a radial distance from the rotational axis given by r_{b}. In some embodiments, the laser may be directed at a location within a ring of radius r_{b} about the rotational axis such that the light reflected from the bottom reflector 1110 is then incident at the top reflector 1112 at a radial distance rₜ from the rotational axis. In such embodiments, light is repeatedly reflected from points in close proximity to the ring of radius rₜ on the top mirror and a ring of radius r_{b} on the bottom reflector 1110.

In some embodiments, laser light may be directed so that the subsequent reflections on the top reflector 1112 have a sequence of radial distances from rotational axis that are bound within a maximum deviation from rₜ. Similarly, in such embodiments, the reflections points on the bottom reflector 1110 may be bound within a maximum deviation from r_{b}. Generally, a laser with transverse spatial extent or diverging/converging angular extent includes laser light filling a bounded extent of radii on the top reflector 1112 and the bottom reflector 1110 after sequential reflections. Embodiments having a larger distribution of the reflection radii may be configured to maximize the time before light is incident on the pump laser injection port (e.g., representing power loss for the laser pump resonator cavity).

In FIG. 14, a projected view is illustrated (e.g., from the top) of rays bouncing from the bottom spherical reflector 1110 to the top spherical mirror 1112. Here, the geometry and rays in this projected horizontal plane are parametrized. The reflection points on the bottom reflector 1110 are at a projected radial distance of r_{b}, and the reflection points on the top reflector 1112 are at a projected radial distance of rₜ. The ray paths for the bottom reflector reflections projected in the horizontal plane make an angle K_{b}, with a radial direction. The ray paths for the top reflector reflections projected in the horizontal plane make an angle Kₜ with a radial direction. The minimum approach distance of the rays to the central axis of the resonator is denoted by the variable a. The horizontally projected ray distance between the reflectors is denoted by the distance w.

FIG. 15 shows the plane of reflection 1122 for rays reflecting from the bottom reflector 1110 in a vertical projection plane that includes the bisector of the incident and reflected rays. Because the plane of reflection includes the surface normal (e.g., as well as the incident and reflected rays), the plane of reflection for a spherical surface also includes the center of curvature 1140 for the spherical surface. The bottom reflector 1110 has a radius of curvature R_{b}, and the top reflector 1112 has a radius of curvature Rₜ. FIG. 14 corresponds to embodiments where the laser is directed to produce reflection rings on the top and bottom resonator mirrors (i.e., 1112 and 1110, respectively) of radius rₜ and r_{b}, respectively. To meet this condition, the normal vectors of the planes of reflection have components in the vertical and radial directions. The distance h is the vertical distance between reflection points on the top reflector 1112 and the reflection points on the bottom reflector 1110. The planes of reflection on the bottom reflector 1110 make an angle α_{b} with the vertical direction 1124. Similarly, the planes of reflection the top reflector 1112 make and angle αₜ with the vertical direction 1124. For the projected perspective of FIG. 15, the horizontal displacement between the reflection point on the bottom reflector 1110 and either the preceding or following reflection points 1108 and 1104 on top reflector 1112 (i.e., point 1120) are separated by a distance w cos (κ_{b}).

The planes of reflection for the bottom and top reflectors 1110 and 1112 are useful to visualize, and it is also useful to define the parameters ρ_{b}=sin(α_{b}) and ρₜ=sin(αₜ). Using similar triangles and inspecting FIG. 15, it can be seen that

$${ρ}_{b} = \frac{{r}_{b}}{{R}_{b}} = \frac{w cos ( {κ}_{b} )}{\sqrt{{w}^{2} {cos}^{2} ( {κ}_{b} ) + {h}^{2}}} , and$$

$${ρ}_{t} = \frac{{r}_{t}}{{R}_{t}} = \frac{w cos ( {κ}_{t} )}{\sqrt{{w}^{2} {cos}^{2} ( {κ}_{t} ) + {h}^{2}}} .$$

Note that the total ray path length between reflections is given by

s=\sqrt{w²+h²}.

Looking at, for example, the line 1124, it can be seen that the portion of the ray distance from the bottom reflection to the point of closest approach to the central axis is

$${Γ}_{b} s = \frac{{r}_{b} cos ( {κ}_{b} )}{w} s ,$$

where is defined Γ_{b} as the proportional “waist” height for the illumination pattern (e.g., not to be confused with beam waist). Similarly, the proportional distance from the top reflection to this waist is given by:

$${Γ}_{t} = \frac{{r}_{t} cos ( {κ}_{b} )}{w} , and$$

$${Γ}_{t} + {Γ}_{b} = 1 .$$

It is convenient to normalize the spatial parameters by the height parameter h, such that

$$\tilde{w} = w / h ,$$

$$\tilde{{r}_{t}} = {r}_{t} / h ,$$

$$\tilde{{r}_{b}} = {r}_{b} / h ,$$

$${\tilde{R}}_{t} = {R}_{t} / h ,$$

$${\tilde{R}}_{b} = {R}_{b} / h ,$$

$${ρ}_{b} = \frac{{r}_{b}}{{R}_{b}} = \frac{\tilde{w} cos ( {κ}_{b} )}{\sqrt{{\tilde{w}}^{2} {cos}^{2} ( {κ}_{b} ) + 1}} , and$$

$${ρ}_{t} = \frac{{r}_{t}}{{R}_{t}} = \frac{\tilde{w} cos ( {κ}_{𝔱} )}{\sqrt{{\tilde{w}}^{2} {cos}^{2} ( {κ}_{t} ) + 1}} .$$

Given this theory, a set of resonator parameters can be calculated as follows:

Select a proportional resonator pattern beam waist ã (e.g., typically based on the expected sampling stream size with the desire to get a relatively good overlap with a small value);

Select a proportional radii \hat{r}ₜ for resonator rings of reflection around the top reflector 1112 typically to match the port radius for the pump laser injection; and

Select the proportional distance from the top reflector 1112 down to the resonator pattern waist: Γ_{b}.

It then follows that Γₜ=1−Γ_{b}.

Then, calculate the horizonal projected reflection angles:

$${κ}_{t} = a sin ( \frac{\tilde{α}}{{\tilde{r}}_{𝔱}} ) .$$

And calculate the horizontal projected ray length between mirror reflections as

$$\tilde{w} = \frac{{\tilde{r}}_{t} cos ( {κ}_{t} )}{{Γ}_{t}} .$$

Then, calculate the actual ray length between mirror reflections as

\tilde{s}=\sqrt{w²+1}.

Calculate the ratio of the top mirror reflection radius to mirror radius of curvature as

$${ρ}_{t} = \frac{{\tilde{r}}_{t}}{{\tilde{R}}_{t}} = \frac{\tilde{w} cos ( {κ}_{t} )}{\sqrt{{\tilde{w}}^{2} {cos}^{2} ( {κ}_{t} ) + 1}}$$

And calculate the top mirror radius of curvature as

$${\tilde{R}}_{t} = \frac{{\tilde{r}}_{t}}{{ρ}_{t}} = {\tilde{r}}_{t} \frac{\sqrt{{\tilde{w}}^{2} {cos}^{2} ( {κ}_{t} ) + 1}}{\tilde{w} cos ( {κ}_{t} )} .$$

Then, calculate the bottom reflection ring radius as

$${\tilde{r}}_{b} = \sqrt{{\tilde{w}}^{2} {Γ}_{b}^{2} + {\tilde{a}}^{2}} .$$

And calculate the bottom mirror radius of curvature

$${\tilde{R}}_{b} = \frac{{\tilde{r}}_{b}}{{ρ}_{b}} = {\tilde{r}}_{b} \frac{\sqrt{{\tilde{w}}^{2} {cos}^{2} ( {κ}_{b} ) + 1}}{\tilde{w} cos ( {κ}_{b} )} .$$

Note that a good design should have subsequent reflections on the top ring that are significantly separated from the location of the laser port to reduce optical loss. The angles per reflection along the top and bottom rings (i.e., 1112 and 1110, respectively) may be calculated as:

$$Δ {θ}_{r} = a sin ( \frac{\tilde{w}}{{\tilde{r}}_{t}} sin ( {κ}_{t} ) ) = a sin ( \frac{\tilde{w}}{{\tilde{r}}_{b}} sin ( {κ}_{b} ) ) .$$

Generally, once a set of parameters is determined, a height h between the top and bottom reflection rings may be selected to scale the selected parameters to desirable values.

For embodiments where the laser injection port is in the top mirror. The distance rₜΔθᵣ should be larger than the radius of the laser port.

### Reverse Calculation

The preceding theory permitted the calculation of the ideal mirror radii of curvature as a function of other system parameters. Suppose though that, after completing the analysis to compute the radii of curvature for the resonator mirrors, one now desires to truncate the values to round numbers for fabrication. With fixed mirror curvatures and finalized “hardware” values for Rₜ,R_{b}, rₜ, r_{b}, the relationship between the remaining parameters a and h can be explored. For example, given the specification of

$${R}_{t} , {R}_{b} , {r}_{t} , {r}_{b} , {ρ}_{b} = \frac{{r}_{t}}{{R}_{t}} and {ρ}_{t} = \frac{{r}_{b}}{{R}_{b}} .$$

This yields relationships between the angles κ_{b}, κₜ and \tilde{w} as:

$${\tilde{w}}^{2} {cos}^{2} ( {κ}_{b} ) = \frac{{ρ}_{b}^{2}}{( 1 - {ρ}_{b}^{2} )} , and$$

$${\tilde{w}}^{2} {cos}^{2} ( {κ}_{t} ) = \frac{{ρ}_{t}^{2}}{( 1 - {ρ}_{t}^{2} )} .$$

This yields the relationship between κ_{b} and κₜ as:

$$\frac{{cos}^{2} ( {κ}_{b} )}{{cos}^{2} ( {κ}_{t} )} = \frac{{ρ}_{b}^{2} ( 1 - {ρ}_{t}^{2} )}{{ρ}_{t}^{2} ( 1 - {ρ}_{b}^{2} )} , or$$

$$\frac{1 - {sin}^{2} ( {κ}_{b} )}{1 - {sin}^{2} ( {κ}_{t} )} = \frac{{ρ}_{b}^{2} ( 1 - {ρ}_{t}^{2} )}{{ρ}_{t}^{2} ( 1 - {ρ}_{b}^{2} )} .$$

Using sin(κ_{b})=a/rₜ and sin(κₜ)=a/r_{b} results in:

$$\frac{1 - {( \frac{a}{{r}_{b}} )}^{2}}{1 - {( \frac{a}{{r}_{t}} )}^{2}} = \frac{{ρ}_{b}^{2} ( 1 - {ρ}_{t}^{2} )}{{ρ}_{t}^{2} ( 1 - {ρ}_{b}^{2} )} ,$$

$$\frac{{r}_{b}^{2} {r}_{t}^{2} - {r}_{t}^{2} {a}^{2}}{{r}_{b}^{2} {r}_{t}^{2} - {r}_{b}^{2} {a}^{2}} = \frac{{ρ}_{b}^{2} ( 1 - {ρ}_{t}^{2} )}{{ρ}_{t}^{2} ( 1 - {ρ}_{b}^{2} )} ,$$

$${r}_{b}^{2} {r}_{t}^{2} - {r}_{t}^{2} {a}^{2} = ( \frac{{ρ}_{b}^{2} ( 1 - {ρ}_{t}^{2} )}{{ρ}_{t}^{2} ( 1 - {ρ}_{b}^{2} )} ) {r}_{b}^{2} {r}_{t}^{2} - ( \frac{{ρ}_{b}^{2} ( 1 - {ρ}_{t}^{2} )}{{ρ}_{t}^{2} ( 1 - {ρ}_{b}^{2} )} ) {r}_{b}^{2} {a}^{2} , and$$

$$( \frac{{ρ}_{b}^{2} ( 1 - {ρ}_{t}^{2} )}{{ρ}_{t}^{2} ( 1 - {ρ}_{b}^{2} )} ) {r}_{b}^{2} {a}^{2} - {r}_{t}^{2} {a}^{2} = ( \frac{{ρ}_{b}^{2} ( 1 - {ρ}_{t}^{2} )}{{ρ}_{t}^{2} ( 1 - {ρ}_{b}^{2} )} ) {r}_{b}^{2} {r}_{t}^{2} - {r}_{b}^{2} {r}_{t}^{2} .$$

This provides the ability to write the modal waist all in terms of known parameters as:

$${a}^{2} = \frac{( [ \frac{{ρ}_{b}^{2} ( 1 - {ρ}_{t}^{2} )}{{ρ}_{t}^{2} ( 1 - {ρ}_{b}^{2} )} ] - 1 ) {r}_{b}^{2} {r}_{t}^{2}}{( [ \frac{{ρ}_{b}^{2} ( 1 - {ρ}_{t}^{2} )}{{ρ}_{t}^{2} ( 1 - {ρ}_{b}^{2} )} ] {r}_{b}^{2} - {r}_{t}^{2} )} .$$

Once the modal waist size is determined, the angles κₜ and κ_{b} can be determined as:

$${κ}_{t} = a sin ( \frac{a}{{r}_{t}} ) , and$$

$${κ}_{b} = a sin ( \frac{a}{{r}_{b}} ) .$$

The parameter \tilde{w}=w/h can then be calculated from:

$${\tilde{w}}^{2} = \frac{{ρ}_{b}^{2}}{{cos}^{2} ( {κ}_{b} ) ( 1 - {ρ}_{b}^{2} )} = \frac{{ρ}_{t}^{2}}{{cos}^{2} ( {κ}_{t} ) ( 1 - {ρ}_{t}^{2} )} , using$$

$$w = {r}_{b} cos ( {κ}_{b} ) + {r}_{t} cos ( {κ}_{t} ) .$$

$$Thus , h = \frac{w}{\tilde{w}} = ( {r}_{b} cos ( {κ}_{b} ) + {r}_{t} cos ( {κ}_{t} ) ) cos ( {κ}_{t} ) \frac{\sqrt{( 1 - {ρ}_{t}^{2} )}}{{ρ}_{t}} ,$$

which is directly calculable from the previous parameters.

### Design for Available Mirror Curvatures

It is convenient to select mirror curvatures that are generally available with minimal customization. Available options would result in a ratio between mirror curvatures. The ratio of the top to bottom mirror radii of curvatures may be given as:

$$\frac{{R}_{t}}{{R}_{b}} = \frac{{\tilde{r}}_{t}}{{\tilde{r}}_{b}} \frac{cos ( {κ}_{b} ) \sqrt{{\tilde{w}}^{2} {cos}^{2} ( {κ}_{t} ) + 1}}{cos ( {κ}_{t} ) \sqrt{{\tilde{w}}^{2} {cos}^{2} ( {κ}_{b} ) + 1}} ; and$$

$$\frac{{R}_{t}}{{R}_{b}} = \frac{{Γ}_{t}}{{Γ}_{b}} \frac{{cos}^{2} ( {κ}_{b} )}{{cos}^{2} ( {κ}_{t} )} \sqrt{\frac{{\tilde{w}}^{2} {cos}^{2} ( {κ}_{t} ) + 1}{{\tilde{w}}^{2} {cos}^{2} ( {κ}_{b} ) + 1}} .$$

$$Then , let ξ = \frac{a}{{r}_{t}} = \frac{\tilde{a}}{{\tilde{r}}_{t}} = sin ( {κ}_{t} ) .$$

$$Using \tilde{w} = \frac{{\tilde{r}}_{t} cos ( {κ}_{t} )}{{Γ}_{t}} and {cos}^{2} ( {κ}_{t} ) = 1 - {sin}^{2} ( {κ}_{t} ) = 1 - {( \frac{α}{{r}_{t}} )}^{2} = 1 - {ξ}^{2} ,$$

$$\frac{{r}_{t}}{{r}_{b}} \frac{cos ( {κ}_{d} )}{cos ( {κ}_{b} )} = \frac{{Γ}_{t}}{{Γ}_{b}} , and {\tilde{w}}^{2} = {( \frac{{\tilde{r}}_{t}}{{Γ}_{t}} )}^{2} ( 1 - {( \frac{a}{{r}_{t}} )}^{2} ) = \frac{( {\tilde{r}}_{t}^{2} - {\tilde{a}}^{2} )}{{Γ}_{t}^{2}} .$$

And the mirror curvature ratios can be calculated as:

$$\frac{{R}_{t}}{{R}_{b}} = \frac{{Γ}_{t}}{{Γ}_{b}} \frac{{Γ}_{b}^{2}}{( 1 - {ξ}^{2} ) {Γ}_{b}^{2} + {ξ}^{2} {Γ}_{t}^{2}} \sqrt{\frac{{( \frac{{\tilde{r}}_{t}}{{Γ}_{t}} )}^{2} {( 1 - {ξ}^{2} )}^{2} + 1}{{( \frac{{\tilde{r}}_{t}}{{Γ}_{t}} )}^{2} ( 1 - {ξ}^{2} ) \frac{( 1 - {ξ}^{2} ) {Γ}_{b}^{2}}{( 1 - {ξ}^{2} ) {Γ}_{b}^{2} + {ξ}^{2} {Γ}_{t}^{2}} + 1}} , or$$

$$\frac{{R}_{t}}{{R}_{b}} = \frac{{Γ}_{t} {Γ}_{b}}{( 1 - {ξ}^{2} ) {Γ}_{b}^{2} + {ξ}^{2} {Γ}_{t}^{2}} \sqrt{\frac{{{\tilde{r}}_{t}^{2} ( 1 - {ξ}^{2} )}^{2} + {Γ}_{t}^{2}}{{\tilde{r}}_{t}^{2} ( 1 - {ξ}^{2} ) \frac{( 1 - {ξ}^{2} ) {Γ}_{b}^{2}}{( 1 - {ξ}^{2} ) {Γ}_{b}^{2} + {ξ}^{2} {Γ}_{t}^{2}} + {Γ}_{t}^{2}}} , or$$

$$\frac{{R}_{t}}{{R}_{b}} = \frac{( 1 - {Γ}_{t} ) {Γ}_{t}}{( 1 - {ξ}^{2} ) {( 1 - {Γ}_{t} )}^{2} + {ξ}^{2} {Γ}_{t}^{2}} \sqrt{\frac{{{\tilde{r}}_{t}^{2} ( 1 - {ξ}^{2} )}^{2} + {Γ}_{t}^{2}}{{\tilde{r}}_{t}^{2} ( 1 - {ξ}^{2} ) \frac{( 1 - {ξ}^{2} ) {( 1 - {Γ}_{t} )}^{2}}{( 1 - {ξ}^{2} ) {( 1 - {Γ}_{t} )}^{2} + {ξ}^{2} {Γ}_{t}^{2}} + {Γ}_{t}^{2}}} .$$

The mirror curvature ratio may be considered a function of the waist ratio ξ, the injection radius may be considered ratio \tilde{r}ₜ, and the proportional waist may be considered location Γₜ. Thus,

$$Z ( {Γ}_{t} , ξ , {\tilde{r}}_{t} ) = \frac{{R}_{t}}{{R}_{b}} = \frac{( 1 - {Γ}_{t} ) {Γ}_{t}}{{( 1 - {Γ}_{t} )}^{2} - ( 1 - 2 {Γ}_{t} ) {ξ}^{2}} \sqrt{\frac{{{\tilde{r}}_{t}^{2} ( 1 - {ξ}^{2} )}^{2} + {Γ}_{t}^{2}}{{\tilde{r}}_{t}^{2} \frac{{( 1 - {ξ}^{2} )}^{2} {( 1 - {Γ}_{t} )}^{2}}{{( 1 - {Γ}_{t} )}^{2} - ( 1 - 2 {Γ}_{t} ) {ξ}^{2}} + {Γ}_{t}^{2}}} .$$

### Limit on Waist Size

It might seem desirable to have the smallest size of beam waist possible (e.g., ξ=0). This is, however, generally undesirable because the rotational symmetry of the system will then return the light without azimuthal rotation about the rotational axis. In the cases where the light is bounded on rings of reflection, the light emitted by the laser injection port that reflects from the bottom mirror (e.g., reflector 1110) will be projected directly back on the laser port and will be lost from the resonator.

Looking at the expression for the reflective rotation,

$${Δθ}_{r} = a sin ( \frac{\tilde{w}}{{\tilde{r}}_{t}} sin ( {κ}_{t} ) ) .$$

Noting that

$$ξ = sin ( {κ}_{t} ) and \frac{\tilde{w}}{{\tilde{r}}_{t}} = \frac{cos ( {κ}_{t} )}{{\tilde{r}}_{t}} = \frac{\sqrt{1 - {ξ}^{2}}}{{Γ}_{t}} , Δ {θ}_{r} = a sin ( \frac{ξ \sqrt{1 - {ξ}^{2}}}{{Γ}_{t}} ) .$$

By selecting a suitable value of Δθᵣ to avoid overlap of the pump beam onto the injection port, the proportional waist size may be solved as follows:

$${ξ}^{2} ( 1 - {ξ}^{2} ) = {Γ}_{t}^{2} {sin}^{2} ( Δ {θ}_{r} ) ,$$

$${ξ}^{4} - {ξ}^{2} + {Γ}_{t}^{2} {sin}^{2} ( Δ {θ}_{r} ) = 0 , and$$

$${ξ}^{2} = \frac{( 1 - \sqrt{1 - 4 {Γ}_{t}^{2} {sin}^{2} ( Δ {θ}_{r} )} )}{2} .$$

For small values of Δθᵣ, an approximate value of ξ may be given as

ξ≈Γₜ sin(Δθᵣ).

Deriving Parameters from Selected Mirror Radii of Curvature

The previous analysis derives radii of curvatures for the pump resonator mirrors. However, it is more useful to select mirror radii of curvatures (ROCs) at rounded numbers and then derive geometric parameters that do not relate to component specifications. If it is instead asserted that the mirror radius curvature be selected,

$${\tilde{r}}_{t}^{2} = {\tilde{R}}^{2} - \frac{1}{4 {( 1 - {ξ}^{2} )}^{2}} ,$$

$${sin}^{2} ( Δ {θ}_{r} ) = 4 {ξ}^{2} ( 1 - {ξ}^{2} ) ,$$

$$4 {ξ}^{4} - 4 {ξ}^{2} + {sin}^{2} ( Δ {θ}_{r} ) = 0 ,$$

$${ξ}^{2} = \frac{1}{2} \pm \frac{1}{2} \sqrt{1 - {sin}^{2} ( Δ {θ}_{r} )} ,$$

$${ξ}^{2} = \frac{1}{2} \pm \frac{1}{2} cos ( Δ {θ}_{r} ) ,$$

$${\tilde{r}}_{t}^{2} = {\tilde{R}}^{2} - \frac{1}{4 {( \frac{1}{2} \pm \frac{1}{2} cos ( Δ {θ}_{r} ) )}^{2}} ,$$

$${\tilde{r}}_{t}^{2} = {\tilde{R}}^{2} - \frac{1}{{( 1 \pm cos ( Δ {θ}_{r} ) )}^{2}} , and$$

$${r}_{t}^{2} = {R}^{2} - \frac{{h}^{2}}{{( 1 \pm cos ( Δ {θ}_{r} ) )}^{2}} .$$

Now suppose the mirrors have diameter D_{R} and the resonator should fit within an integrating volume of radius Rₛ, the height of the integrating volume at the edge of the resonator mirrors may be given as

$${H}_{S} = 2 ( \sqrt{{R}_{S}^{2} - {( \frac{{D}_{R}}{2} )}^{2}} ) ,$$

whereas the height between the resonating mirror surfaces at the edge of the mirrors may be given as

$${H}_{M} = 2 ( \frac{h}{2} - \sqrt{{R}_{t}^{2} - {r}_{t}^{2}} + \sqrt{{R}_{t}^{2} - {( \frac{{D}_{R}}{2} )}^{2}} ) .$$

The sum of edge distances between the mirror and the interior of the integrating volume may then be given as

$$d = {H}_{S} - {H}_{M} = - h + 2 ( \sqrt{{R}^{2} - {r}_{t}^{2}} + \sqrt{{R}_{S}^{2} - {R}_{M}^{2}} - \sqrt{{R}^{2} - {R}_{M}^{2}} ) ,$$

$$d = h ( \frac{2}{( 1 + cos ( Δ {θ}_{r} ) )} - 1 ) + 2 ( \sqrt{{R}_{S}^{2} - {R}_{M}^{2}} - \sqrt{{R}^{2} - {R}_{M}^{2}} ) ,$$

$$d = h ( \frac{1 - cos ( Δ {θ}_{r} )}{1 + cos ( Δ {θ}_{r} )} ) + 2 ( \sqrt{{R}_{S}^{2} - {R}_{M}^{2}} - \sqrt{{R}^{2} - {R}_{M}^{2}} ) , and$$

$$h = [ d - 2 ( \sqrt{{R}_{S}^{2} - {R}_{M}^{2}} - \sqrt{{R}^{2} - {R}_{M}^{2}} ] ( \frac{1 + cos ( Δ {θ}_{r} )}{1 + cos ( Δ {θ}_{r} )} ) .$$

If d, Rₛ, R, Dᵣ, Δθᵣ are chosen, h can be calculated as

$$h = [ d - 2 ( \sqrt{{R}_{S}^{2} - {R}_{M}^{2}} - \sqrt{{R}^{2} - {R}_{M}^{2}} ] ( \frac{1 + cos ( Δ {θ}_{r} )}{1 + cos ( Δ {θ}_{r} )} ) , and$$

$${r}_{t}^{2} = {R}^{2} - \frac{{h}^{2}}{{( 1 \pm cos ( Δ {θ}_{r} ) )}^{2}} , with an injection angle$$

$${κ}_{t} = a cos ( \sqrt{\frac{1}{2} ( 1 + cos ( Δ {θ}_{r} ) )} ) , with a waist distance$$

$$a = {r}_{t} \sqrt{\frac{1}{2} ( 1 + cos ( Δ {θ}_{r} ) )} , and a projected ray length of$$

$$w = 2 {r}_{t} cos ( {κ}_{b} ) .$$

Note that in this calculation, d and Δθᵣ may be “judgement calls”, but the other parameters may be limited by available hardware. This approach limits the parameter search for the design significantly.

### Vertically Symmetric Systems

Placing the waist of the pump resonator pattern at specific locations between the resonator mirrors may have specific value to optimize the pump laser overlap with an analyte flow. For example, if the radial distribution of the flow is increasing as it flows between the resonator mirrors (e.g., reflectors 1110 and 1112), the optical overlap with the analyte may be improved by placing the resonator light field waist closer to the analyte exit port.

However, if the analyte radial distribution is uniform as it flows between the pump resonator reflectors, a symmetric system placing the light field waist halfway between the resonator reflectors may be a preferred solution. In this special case, Γₜ=Γ_{b}=0.5. The reflectors radii of curvature may be the same for the top reflector (e.g., reflector 1112) and the bottom reflector (e.g., reflector 1110), and

$$Z ( 1 / 2 , ξ , {\tilde{r}}_{t} ) = \frac{{R}_{t}}{{R}_{b}} = \sqrt{\frac{4 {{\tilde{r}}_{t}^{2} ( 1 - {ξ}^{2} )}^{2} + 1}{4 {{\tilde{r}}_{t}^{2} ( 1 - {ξ}^{2} )}^{2} + 1}} = 1 .$$

In this special symmetric case, proportional mirror radii of curvature are a function of the laser port injection radius and the waist size:

$${\tilde{R}}_{t} = {\tilde{R}}_{b} = {\tilde{r}}_{b} \frac{\sqrt{{\tilde{w}}^{2} {cos}^{2} ( {κ}_{b} ) + 1}}{\tilde{w} cos ( {κ}_{b} )} = \sqrt{{\tilde{r}}_{t}^{2} + \frac{1}{4 {( 1 - {ξ}^{2} )}^{2}}} .$$

For small values of ξ²

$${\tilde{R}}_{t} = {\tilde{R}}_{b} \approx \sqrt{{\tilde{r}}_{t}^{2} + \frac{1}{4}} \sqrt{1 + \frac{{ξ}^{2}}{2 ( {\tilde{r}}_{t}^{2} + \frac{1}{4} )}} \approx ( 1 + \frac{{ξ}^{2}}{( 4 {\tilde{r}}_{t}^{2} + 1 )} ) \sqrt{{\tilde{r}}_{t}^{2} + \frac{1}{4}} .$$

In the limit of a vanishing waist size,

$${\tilde{R}}_{t} = {\tilde{R}}_{b} = \sqrt{{\tilde{r}}_{t}^{2} + \frac{1}{4}} .$$

However in that limit, Δθᵣ=0, the laser beam would be reflected directly back on the laser port.

### Pump Resonator Stability

The geometric description above related to a central ray that reflects between a top reflection ring and a bottom reflection ring (e.g., of the reflectors 1112, and 1110, respectively). However, an injected laser beam has a distribution of directions about the central laser path. In many embodiments, it is desirable that rays reflecting from the top and bottom mirrors in close proximity to the reflection rings of radius rₜ and r_{b} remain in close proximity to those reflection rings after subsequent reflections to maintain a relatively tight waist in the resulting light field.

Using conventional resonator stability analysis, the ideal central laser rays between the reflections can be unwrapped. This reflective path should be approximately periodic in its effect on the beam surrounding the central beam. The ABCD matrix for ray-tracing for the path from the top mirror to the bottom mirror and back to the top mirror may be written as follows:

$${M}_{A B C D} = [ \begin{matrix}1 & 0 \\ - 2 / {R}_{t} & 1\end{matrix} ] [ \begin{matrix}1 & s \\ 0 & 1\end{matrix} ] [ \begin{matrix}1 & 0 \\ - 2 / {R}_{b} & 1\end{matrix} ] [ \begin{matrix}1 & s \\ 0 & 1\end{matrix} ] = [ \begin{matrix}1 - \frac{2 s}{{R}_{b}} & s ( 2 - \frac{2 s}{{R}_{b}} ) \\ - \frac{2}{{R}_{t}} - \frac{2}{{R}_{b}} + - \frac{4 s}{{R}_{b} {R}_{t}} & ( 1 - \frac{2 s}{{R}_{t}} ) ( 1 - \frac{2 s )}{{R}_{b}} ) - \frac{2 s}{{R}_{t}}\end{matrix} ] .$$

The cavity stability requirement is the determinate of this matrix being between 0 and 1, which is a classic resonator stability criteria, written as:

$$0 \leq ( 1 - \frac{s}{{R}_{b}} ) ( 1 - \frac{s}{{R}_{t}} ) = {g}_{2} {g}_{1} \leq 1 ,$$

$${g}_{1} = ( 1 - \frac{\sqrt{{\tilde{w}}^{2} + 1}}{{\tilde{R}}_{t}} ) = ( 1 - \frac{\sqrt{{\tilde{r}}_{t}^{2} ( 1 - {ξ}^{2} ) + {Γ}_{t}^{2}}}{{\tilde{R}}_{t} {Γ}_{t}} ) , and$$

$${g}_{2} = ( 1 - \frac{\sqrt{{\tilde{w}}^{2} + 1}}{{\tilde{R}}_{b}} ) = ( 1 - \frac{\sqrt{{\tilde{r}}_{𝔱}^{2} ( 1 - {ξ}^{2} ) + {Γ}_{𝔱}^{2}}}{{\hat{R}}_{b} {Γ}_{t}} ) .$$

For the special case of Γ_{b}=Γₜ=0.5,

$${g}_{1} = {g}_{2} = ( 1 - \frac{\sqrt{4 {\tilde{r}}_{t}^{2} ( 1 - {ξ}^{2} ) + 1}}{{\tilde{R}}_{t}} ) \approx ( 1 - 2 ( 1 - 2 {\tilde{r}}_{t}^{2} \frac{{ξ}^{2}}{( 4 {\tilde{r}}_{t}^{2} + 1 )} ) ( 1 - \frac{{ξ}^{2}}{( 4 {\tilde{r}}_{t}^{2} + 1 )} ) ) .$$

Generally, small values of ξ, g₁g₂ may be bounded between 0 and 1 for a stable cavity.

FIG. 16 shows one exemplary embodiment of a sensor system 1200 utilizing a 4″ LabSphere™ integrating sphere 1202 and a Thorlabs™ coded aperture spectrometer 1208. The integrating sphere 1202 is fitted for air flow, containing particulates, through the top at the inlet 1216, clean air for protective sheath air through the side inlet 1212, which passes through an exterior coaxial cylindrical nozzle surrounding the nozzle connected to inlet 1216, and a laser injection port 1214 on the top. The bottom mirror (not shown here as it is inside the sphere 1202) and analyte exhaust port 1204 is on the bottom. The laser light is propagated through the top mirror (not shown here as it is also inside the sphere 1202) to the bottom mirror. A threaded fixture may permit fine tuning of the mirror separation by displacements of the bottom mirror during fabrication.

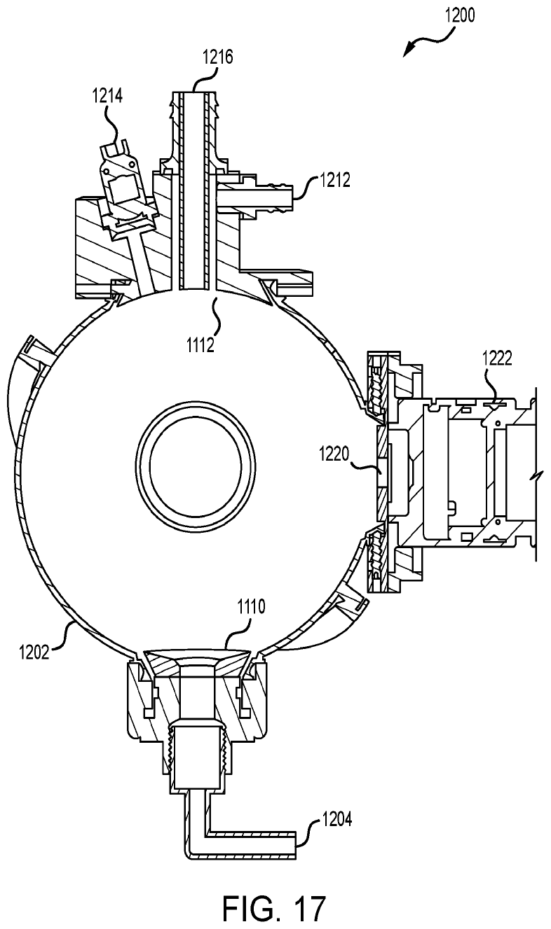

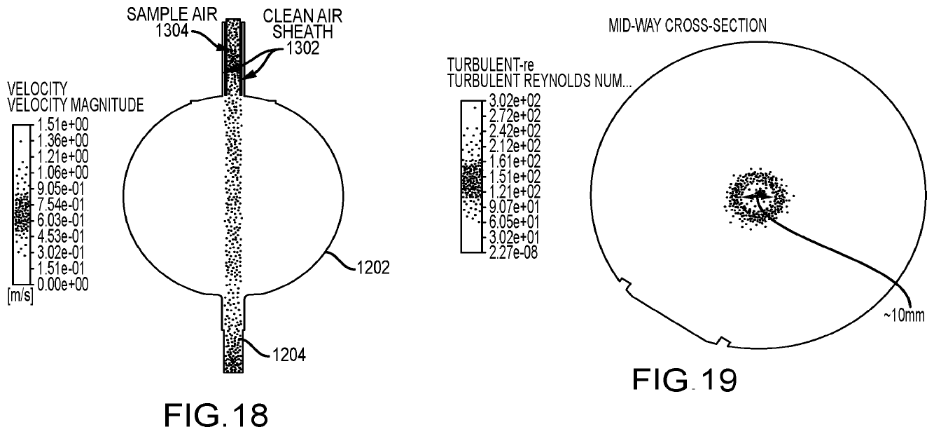

FIG. 17 is a cross-sectional view of the sensor system 1200 with the analyte injection inlet 1216, the pump laser injection port 1214, and a spectrometer fixture 1222 coupled to the optical aperture 1220, in one exemplary embodiment. The laser 1214, in this embodiment, propagates the laser light to the bottom mirror 1110 which reflects to the top mirror 1112. The resonator mirrors 1112 and 1110 provide laser pump resonator within the integrating sphere 1202. The flow of the combined sheath air and analyte stream, as computed by CFD (computational fluid dynamics) analysis, are shown below in FIGS. 18 and 19, in one exemplary embodiment. The nozzle-in-nozzle design matches the flow to minimize mixing and turbulent flow, while surrounding the analyte with clean air. This design limits the accumulation of analyte on optical surfaces, prolonging the operation of the device without cleaning or maintenance. The flow stream may expand slightly between the entry and exhaust ports and may have a size that is sufficiently large to overlap the pump mode.

The flow of the particle flow 1304, in this embodiment, is designed for 1 Liter/min through the particle flow nozzle. The particle nozzle, in this embodiment, has an exterior diameter of 5/16″ (7.94 mm) with an interior diameter of 0.241″ (6.12 mm). The sheath air nozzle, in this embodiment, has a diameter of 13 mm. The flow of the sheath air 1302 is designed to match the velocity of the particle flow 1304. As shown in FIGS. 18 and 19, based on CFD analysis, the flows mesh together in a very short distance and generally do not spread much at the bottom reflector. A 1.5 mm×45° chamfer is generally enough to direct the exhaust into a 10 mm exhaust port 1204.

FIGS. 18 and 19 show a 2D slice of the Reynolds number of the flow at the center of the integrating volume, in one exemplary embodiment. At the center of the integrating volume, the particle flow is approximately 10 mm diameter. The Reynolds number of the flow is generally very low (e.g., below 300), and should be able to maintain laminar flow. This is important because, should something happen where the flows get disrupted due to an obstruction in the flow tubing, it should be possible to keep the flow laminar.

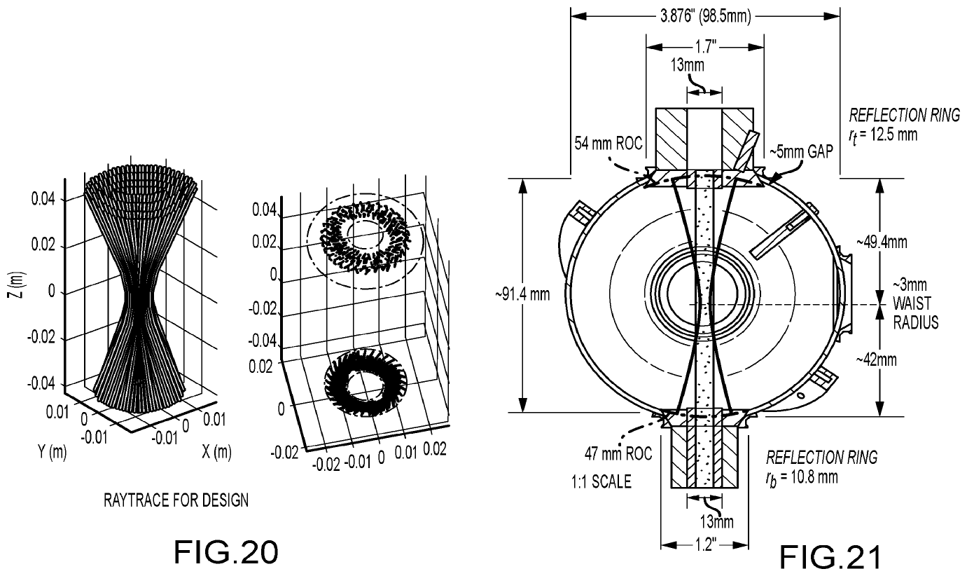

FIGS. 20 and 21 illustrate a pump resonator design, in one exemplary embodiment. The pump mode resonator in this embodiment has been designed to maintain a pump light on surfaces of a 1.7″ diameter top mirror and 1.2″ diameter bottom mirror. The accumulated laser pump resonator beam shape is an hourglass shape with a hole in the middle and has a waist radius of approximately 3 mm. The design has been made to place the pump field waist slightly below the center of the integrating volume to take advantage of the enlargement of the analyte flow radius closer to the exhaust port. The top and bottom resonator mirrors (e.g., reflectors 1112 and 1110, respectively) are configured with central holes that are 13 mm in diameter and 10 mm, respectively, with a 1.5 mm×45° chamfer to accommodate the analyte sheath flow and exhaust. The top resonator mirror has been designed with a 54 mm radius of curvature and the bottom mirror has been designed with a 42 mm radius of curvature. The vertical distance between the designed “reflection rings” on the top and bottom mirror are approximately 91.4 mm, and the laser is injected on the top mirror at a radius of 12.5 mm relative to the axis of symmetry.

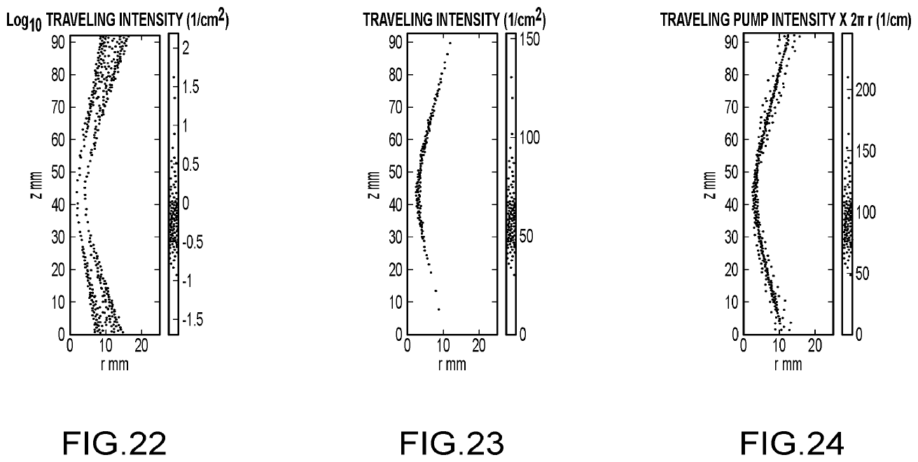

FIGS. 22-24 illustrate pump resonator intensity profiles, in some exemplary embodiments. With the design configurations just mentioned, the simulated intensity distributions are shown with arbitrary relative units having an injection power of “1”. A graph of traveling normalized intensity times 2xr is shown as an appropriate unit for axially symmetric power flow.

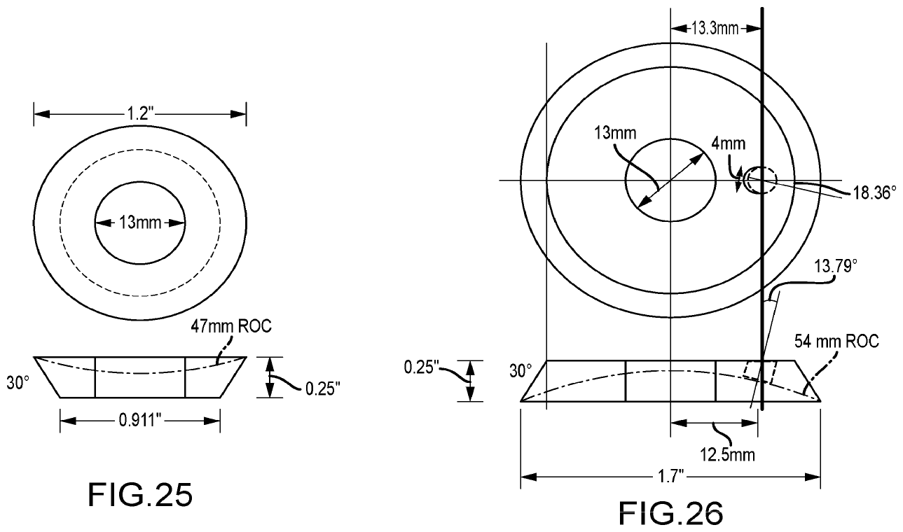

FIGS. 25 and 26 illustrate mirror design specifications of the top and bottom mirrors (e.g., reflectors 1112 and 1110, respectively), in some exemplary embodiments. The specified polar azimuthal angle for the injected laser beam direction, in this embodiment, is smaller at the back surface of the top mirror, because the hole in the back of the mirror should be placed at a larger radius than the hole exits on the reflective surface. The results include a reduced azimuthal angle.

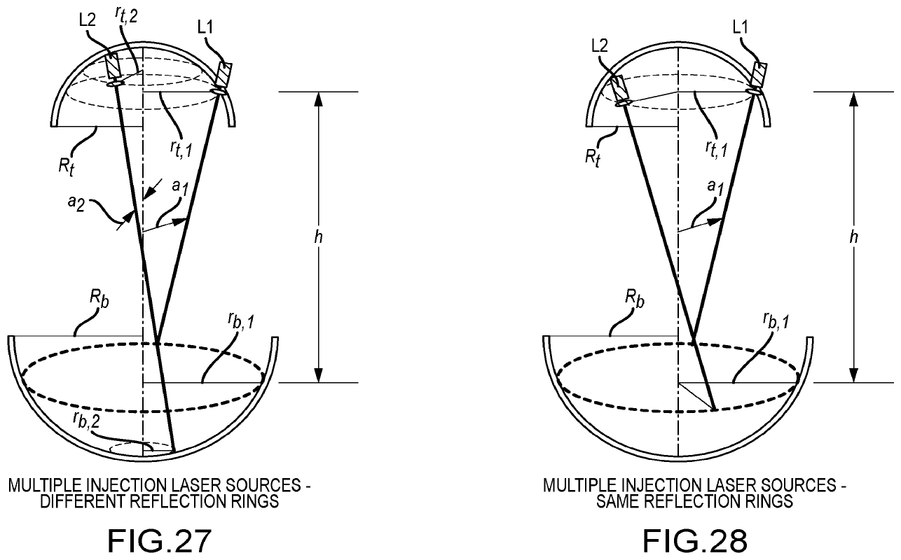

FIG. 27 illustrates multiple laser injections with reflections between different reflection rings, and FIG. 28 illustrates multiple laser injections with reflections between same reflection ring, in some exemplary embodiments. Many of the embodiments discussed above utilize a single injection laser. The embodiments herein, however, are not intended to be limited to such. For example, the selected pump resonator mirrors may support injection lasers from many possible positions. Multiple lasers may be injected at the same reflection ring radii (e.g., rₜ on the top mirror or r_{b} on the bottom mirror) but from different azimuthal angles. Alternatively or additionally, different pump lasers may be injected at different calculated reflection ring radii corresponding to different waist parameters ‘a’ or Γₜ parameters. While additional injection ports may result in additional resonator loss locations, additional laser powers offer opportunities to scale injected laser power, select different pumping wavelengths, or even utilize scattering processes involving pump/probe excitation or pumping from multiple simultaneous wavelengths.

FIGS. 27 and 28 show examples with injection locations from laser L1 and laser L2. In these embodiments, the lasers L1 and L2 illustrate the injected beam path from top reflection rings to bottom reflection rings (e.g., of the reflectors 1112 and 1110, respectively). In FIG. 27, the laser injection location and angle of L2 may be tied to the reflection rings r_{b,2} and r_{t,2}, whereas the laser injection location and angle of L1 may be tied to the reflection rings r_{b}, 1 and r_{t,1}. In FIG. 28, both L1 and L2 are injected from the same top reflection ring r_{t,1} and are directed toward the bottom reflection ring r_{b,1}. Thus, the reflection rings may be considered as theoretical constructs. And the beam extents of the two lasers L1 and L2 may have substantial spatial overlap on the mirrors even if the two lasers are using different sets of reflection rings.

In embodiments having multiple laser injection ports, multiple lasers may have different wavelengths and be configured so that a single laser may be operated at a time to collect multiple spectra from multiple pump sources. In some embodiments, one source may be used simply to sense Rayleigh scattering from a particulate and trigger higher power pumps for spectral sensing. This approach can be used to reserve laser power only for when particulates are in the integrating volume and reduce the power consumption of the system. If a single laser can operate in both a high power and a low power mode, this same technique could be used without requiring multiple lasers. Alternatively or additionally, the pump resonator may use one wavelength to collect shorter frequencies while another may collect longer frequencies of Raman scattering in order to measure with higher resolution spectra from a single detector.

In some embodiments, the injection laser port may include a window to maintain a lower internal pressure of the integrating volume. In embodiments utilizing polarized lasers sources, the window may be selected and configured for high polarization transmission from its associated laser source. The window may also be selected for higher reflectivity at polarizations other than the laser injection polarization. The laser polarization, after multiple bounces within the integration volume, may undergo rotation so that the transmission through the injection window as a source of resonator loss is reduced.

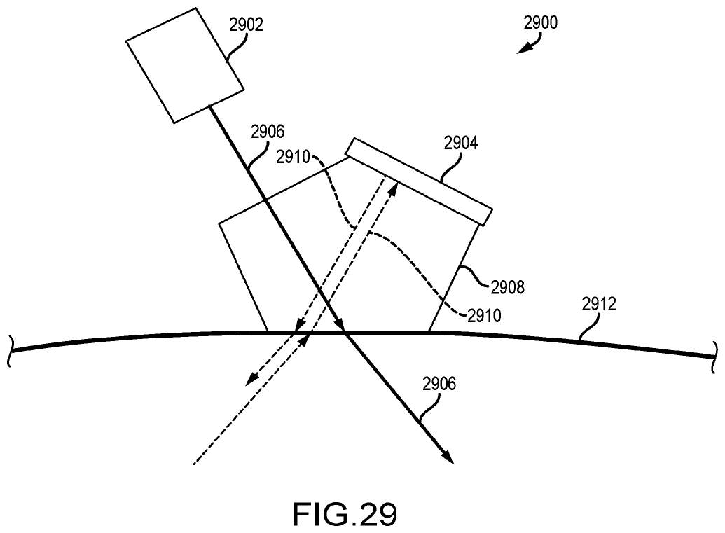

FIG. 29 illustrates a block diagram of a pump laser injection prism system 2900, in one exemplary embodiment. Here, the laser injection assembly may include a prism 2908 and a mirror 2904 that are designed to reflect pump light 2910 entering the prism 2908 from the integrating volume 2912. The incident light angles from pump light paths 2910 are generally near mirrors of the light paths of the injected light direction (i.e., of the laser light 2906 from the laser 2902) about a known designed plane. A surface of the prism may include a reflective coating 2904 to reflect the light 2910 back into the integrating volume into a known pump resonator mode. In some variants of this embodiment, pump laser light may eventually return on the same path as it was initially injected and the laser cavity itself may be bidirectionally coupled to the laser pump resonator to improve the overall efficiency of the laser pump resonator.

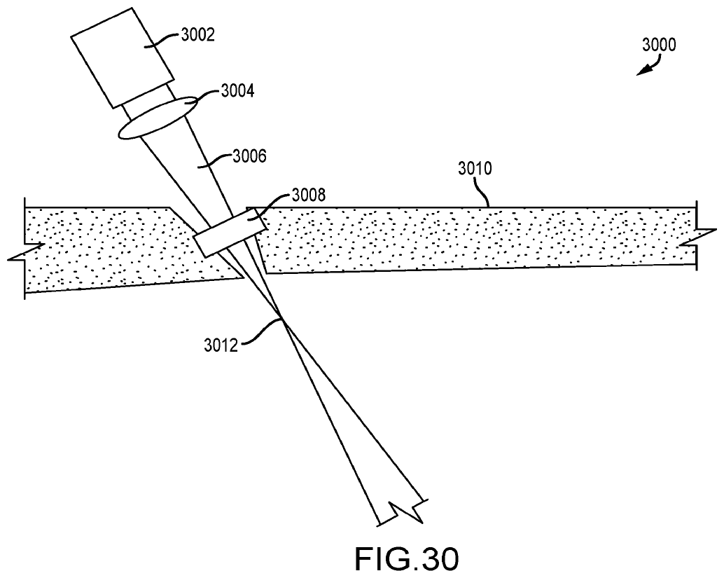

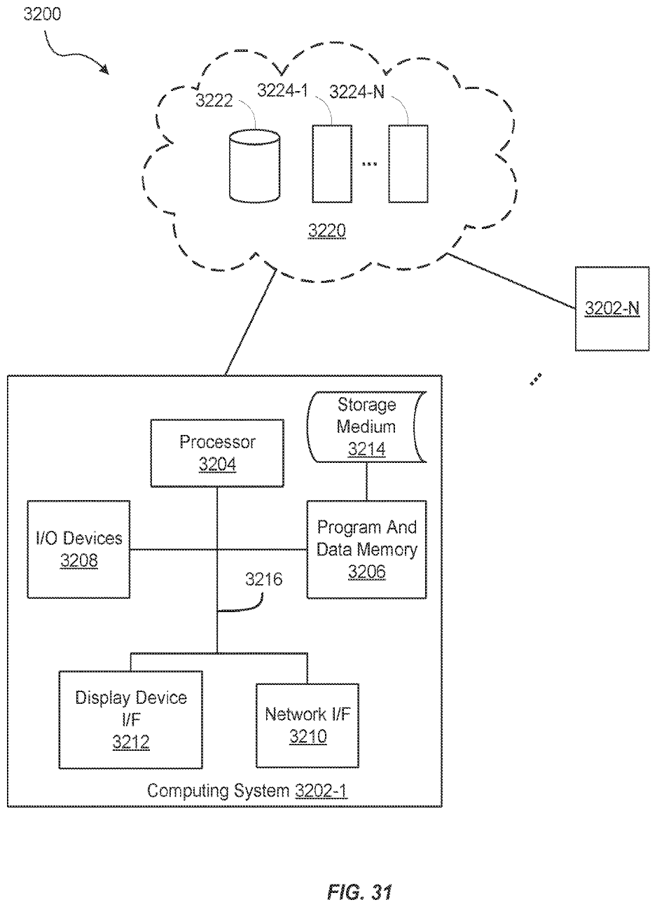

FIG. 30 illustrates a block diagram of a laser pump resonator 3000 converging a laser 3002 to reduce injection laser aperture on mirrored surface, in one exemplary embodiment. Here, the laser light 3006 may be focused via a lens 3004 to a small beam waist 3012 in close proximity to the injection aperture 3008 on the mirrored surface of a pump resonator mirror 3010. This approach can be used to permit a small injection port aperture on the mirrored surface and reduce the loss of pump resonator light that may overlap the injection aperture on subsequent laser bounces within the resonator.

The objective of the laser pump resonator is to create a distribution of light that maximizes overlap with an analyte stream. For the purpose of analysis, it can be assumed that the stream has a Gaussian distribution of radius b. Then, an overlap integral of over a ray length can be calculated with the analyte density as a weighting given a minimum distance to the center of the distribution given by a.

The integral may be given by

$$M = \frac{\sqrt{{w}^{2} + {h}^{2}}}{w} {\int }_{∞}^{∞} exp ( - \frac{2 {r}^{2}}{{b}^{2}} ) d l ,$$

which may be worked out as

$$M ( a , b ; h , w ) = \sqrt{\frac{π}{2}} \sqrt{1 + {( h / w )}^{2}} ( b exp ( - \frac{2 {a}^{2}}{{b}^{2}} ) ) .$$

The overlap integral may be maximized when a=0 and b is as large as possible. However, as discussed before, a may be limited by the requirement to avoid having the beam retroreflected directly back on to the pump injection port.

The embodiments here are not intended to be limited to a specific selection of optical spectrometer, however spectrometers supporting higher throughput (etendue) are generally desirable. A spectrometer technology that may be particularly well suited to embodiments herein includes a coded aperture spectrometer. A coded aperture spectrometer uses a patterned aperture and computational processing of a spectrally dispersed illuminated aperture. Because the physical size of the aperture can be substantially larger than a single slit, more light can be collected by the spectrometer. Overall, the spectrometer's etendue is much larger than many other spectrometer technologies.

Additionally, while the embodiments herein may be used for spectral analysis of Raman scattering from analytes, other embodiments may be constructed for sensing other scattering or reemission processes. For example, laser pumping can result in fluorescence of analytes and the resonant pumping, the integrating volume, and the spectrometer may be constructed for sensitive spectral analysis of fluorescence. Likewise, in some embodiments, illumination from multiple pump laser sources at different wavelengths can be used for spectrometer sensing of nonlinear or multi-photon scattering processes.

Moreover, because Rayleigh scattering intensity is generally orders of magnitude stronger than Raman scattering intensity, it is common for a spectrometer to include a blocking filter (e.g., a long pass filter) to block light at the laser pump wavelength and reduce optical scatter and the resulting signal contamination at the scattered emission wavelengths. In this regard, a detector may be included within the optical configuration of the system to detect the pump wavelength and to detect the presence of particulates from Mie or other scattering processes. When scattering events are scarce, Rayleigh scattering of particulates may be used to trigger pump laser pulsing at higher powers than would be available as continuous sources. Thus, in some embodiments, a detector of Rayleigh scattering may be integrated with the spectrometer before the pump wavelength is filtered, or through an additional port on the integrating volume.

In some embodiments, the amount of recorded Raman scatter from any single particle may not be enough to overcome the inherent noise in the spectrometer, so spectral data may be collected by integrating a signal over a period of many seconds. Automated detection modules can monitor the recorded signal and adjust the integration time to incorporate shorter integration times with multiple measurements averaged together. Alternatively, a single longer integration may be employed to maximize the signal-to-noise ratio and improve measurement for detecting the chemical aerosol. In any case, at the end of each time period, the recorded Raman spectrum is processed.

In some embodiments, the sensors described herein may be utilized as a real-time monitor for chemical aerosol plumes over time. For example, by adding tailored pre-processing, conditioning, and multi-time-scale background subtraction techniques, the sensor can track when a new chemical is present or leaves returning to the original background. Monitoring can be accomplished using a signature library approach (e.g., a database for comparison of detections to known results) or a library-free approach.

For the signature library approach, after each measurement, spectroscopic data processing techniques can be applied to separate a measured mixture of chemicals into individual measured components. The separation of components includes a library of known chemical components. If a mixture of measurements into chemical components cannot be fully separated (e.g., because there are unmatched spectral vibrational bands), it is possible that a compound unknown to the library was found. This compound spectrum could be added as another component in the spectral library for future use. For scenarios where the background aerosol composition does not change rapidly, the sensor embodiments herein may use consistent spectral features from previous or future measurements as a background spectrum. Removing or using a premeasured background spectrum in processing may be useful in separating the chemical spectrum of the chemical aerosol from the measured mixture.

For spectral sensor embodiments using the library-free approach, deep neural network techniques may be used to relate measured spectra to molecular structures. For example, using the SMILES (Simplified molecular-input line-entry system) text-based representation of a molecular structure, the sensor embodiments herein can apply a variational autoencoder to learn a latent space that encodes molecule structure, and then develop a model that relates the latent space to the spectral signature.

In some embodiments, the sensor may be used as a targeted detection sensor. Such may be a case where a chemical is suspected to be present within the air. For example, there may not be a known or premeasured background. However, the sensor may use a processing algorithm to focus on separating measured spectra into individual chemical species.

In some embodiments, the spectrometer could be used for measuring a liquid fluid as it passes through the integrating volume. For example, a transparent tube could be used in replacement of the particle nozzle and pass through both reflectors. Sheath flow may not be necessary as the sample flow would be contained. The pump resonator computations would account for the refraction of the laser light as it transmits through the tube walls. Alternatively or additionally, issues with refraction from the transparent tube could be avoided by having the inner volume of the integrating volume filled with a material which matches the refractive index of the tube walls. Specifically, for Raman spectral sensing, it may be necessary to find a material which also is also a low Raman scatterer so as to not overshadow the Raman emission of the liquid sample.

Any of the above embodiments herein may be rearranged and/or combined with other embodiments. Accordingly, the concepts herein are not to be limited to any particular embodiment disclosed herein. Additionally, the embodiments can take the form of entirely hardware or comprising both hardware and software elements. Portions of the embodiments may be implemented in software, which includes but is not limited to firmware, resident software, microcode, etc. FIG. 31 illustrates a computing system 3200 in which a computer readable medium 3206 may provide instructions for performing any of the methods disclosed herein.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by a user. Cloud computing relies on the sharing of resources, and generally includes on-demand self-service, broad network access, resource pooling, rapid elasticity, and measured service.

In FIG. 31, one illustrative cloud computing system 3200 is illustrated and is operable to perform the above operations by executing programmed instructions tangibly embodied on one or more computer readable storage mediums. The cloud computing system 3200 generally includes the use of a network of remote servers hosted on the internet to store, manage, and process data, rather than a local server or a personal computer (e.g., in the computing systems 3202-1-3202-N). Cloud computing enables users to use infrastructure and applications via the internet, without installing and maintaining them on-premises. In this regard, the cloud computing network 3220 may include virtualized information technology (IT) infrastructure (e.g., servers 3224-1-3224-N, the data storage module 3222, operating system software, networking, and other infrastructure) that is abstracted so that the infrastructure can be pooled and/or divided irrespective of physical hardware boundaries. In some embodiments, the cloud computing network 3220 can provide users with services in the form of building blocks that can be used to create and deploy various types of applications in the cloud on a metered basis.

Various components of the cloud computing system 3200 may be operable to implement the above operations in their entirety or contribute to the operations in part. For example, a computing system 3202-1 may be used to perform all or portions of the spectral analysis and/or the sensor operations, and then store that analysis in a data storage module 3222 (e.g., a database) of a cloud computing network 3220. Various computer servers 3224-1-3224-N of the cloud computing network 3220 may be used to operate on the data and/or transfer the analysis and/or the data to another computing system 3202-N.

Some embodiments disclosed herein may utilize instructions (e.g., code/software) accessible via a computer-readable storage medium for use by various components in the cloud computing system 3200 to implement all or parts of the various operations disclosed hereinabove. Examples of such components include the computing systems 3202-1-3202-N.

Exemplary components of the computing systems 3202-1-3202-N may include at least one processor 3204, a computer readable storage medium 3214, program and data memory 3206, input/output (I/O) devices 3208, a display device interface 3212, and a network interface 3210. For the purposes of this description, the computer readable storage medium 3214 comprises any physical media that is capable of storing a program for use by the computing system 3202. For example, the computer-readable storage medium 3214 may be an electronic, magnetic, optical, electromagnetic, infrared, semiconductor device, or other non-transitory medium. Examples of the computer-readable storage medium 3214 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 3204 is coupled to the program and data memory 3206 through a system bus 3216. The program and data memory 3206 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 3208 (including but not limited to keyboards, displays, touchscreens, microphones, pointing devices, etc.) may be coupled either directly or through intervening I/O controllers. Network adapter interfaces 3210 may also be integrated with the system to enable the computing system 3202 to become coupled to other computing systems or storage devices through intervening private or public networks. The network adapter interfaces 3210 may be implemented as modems, cable modems, Small Computer System Interface (SCSI) devices, Fibre Channel devices, Ethernet cards, wireless adapters, etc. Display device interface 3212 may be integrated with the system to interface to one or more display devices, such as screens for presentation of data generated by the processor 3204.

What is claimed is:

1. A sensor, comprising:
a spectrometer;
an integrating volume comprising a laser pump resonator configured within the integrating volume, an interior reflective surface, and an inlet operable to receive a flow of an analyte;
a laser operable to excite the analyte with laser light via the laser pump resonator to generate analyte signature light from the excited analyte; and
an optical port configured with the integrating volume, and operable to direct the analyte signature light from the integrating volume to the spectrometer during reflections of the laser light in the laser pump resonator,
wherein the spectrometer is operable to identify the analyte based on the analyte signature light.

2. The system of claim 1, wherein:
the integrating volume comprises an exhaust port operable to exhaust the analyte from the sensor.

3. The sensor of claim 2, further comprising:
a vacuum device coupled to the exhaust port to maintain a pressure lower than an external environment.

4. The sensor of claim 1, further comprising:
a nozzle configured with the integrating volume and operable to provide a sheath of clean gas surrounding the flow of the analyte.

5. The sensor of claim 1, wherein:
the spectrometer is a coded aperture spectrometer.

6. The sensor of claim 1, wherein:
the optical port comprises an afocal optical system operable to direct the signature light to the spectrometer.

7. The sensor of claim 1, wherein the laser pump resonator comprises:
a first resonator mirror and a second resonator mirror,
wherein the first resonator mirror includes at least one laser entrance port,
wherein the second resonator mirror includes an exhaust port operable to exhaust the flow of the analyte, and
wherein the first and second resonator mirrors are operable to resonate the laser light within the laser pump resonator to excite and scatter the analyte signature light inside the integrating volume.

8. The sensor of claim 7, wherein:
the first and second resonator mirrors are configured to substantially confine the laser light within the laser pump resonator.

9. The sensor of claim 7, wherein:
the first and second resonator mirrors are concave.

10. The sensor of claim 7, wherein:
each of the first and second resonator mirrors comprises a spherical specular reflective surface.

11. The sensor of claim 7, wherein:
the laser pump resonator further comprises an optical port operable to propagate the laser light into the laser pump resonator.

12. The sensor of claim 11, wherein:
the other optical port is configured to propagate the laser light to approximately a center of the flow of the analyte from an acute angle with respect to the inlet.

13. The sensor of claim 1, wherein:
the interior reflective surface comprises a first mirror with an ellipsoidal shape and a second mirror with a spherical shape.

14. The sensor of claim 13, wherein:
the second mirror comprises a diffusely scattering patch of material.

15. The sensor of claim 1, wherein:
the interior reflective surface internally reflects with specular reflection.

16. The sensor of claim 1, further comprising:
optically reflective fairings operable to block light from entering the integrating volume from at least one of the inlet, or the optical port.

17. The sensor of claim 1, further comprising:
a data library comprising data sets of analytes for comparison to spectral data from the spectrometer to identify the analytes.

18. The sensor of claim 1, further comprising:
another laser configured to excite the analyte with other laser light in the laser pump resonator.

19. The sensor of claim 17, wherein:
the other laser light comprises a different wavelength than the laser light.

20. A method of identifying an analyte comprises:
flowing air comprising the analyte into an integrating volume that includes a laser pump resonator configured within the integrating volume;
exciting the analyte with laser light via the laser pump resonator to generate analyte signature light from the excited analyte;
directing the analyte signature light from the integrating volume to a spectrometer via at least one reflection from an internal reflective surface of the integrating volume during reflections of the laser light in the laser pump resonator; and
identifying the analyte based on the analyte signature light.
